(19) United States
(12) Patent Application Publication
(10) Pub. No.: US 2007/0173438 A1
(43) Pub. Date: Jul. 26, 2007

(54) [PSI[CH2NH]PG4] glycopeptide antibiotic analogs

(75) Inventor: Dale L. Boger, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 11/654,466

(22) Filed: Jan. 16, 2007

Related U.S. Application Data
(60) Provisional application No. 60/759,110, filed on Jan. 13, 2006.

(51) Int. Cl. A61K 38/14

(52) U.S. Cl. 514/8

(57) ABSTRACT

[ψ[CH₂NH]PG⁴] glycopeptide antibiotic analogs are reengineered forms of glycopeptides that exhibit antimicrobial activity against both wild type and glycopeptide antibiotic resistant strains of microorganisms. For example, [ψ[CH₂NH]Tpg⁴] vancomycin aglycon is a reengineered form of vancomycin that exhibits antimicrobial activity (MIC=31 μg/mL) against both wild type and VanA resistant organism (E. faecalis BM4166). The VanA resistant organism achieves its resistance, upon glycopeptide antibiotic challenge, by remodeling its D-Ala-D-Ala peptidoglycan cell wall precursor to D-Ala-D-Lac. [ψ[CH₂NH]PG⁴] glycopeptide antibiotic analogs have an altered glycopeptide backbone wherein the carbonyl of the fourth amino acid residue of the glycopeptide backbone has been replaced with a methylene. This alteration of the glycopeptide backbone imparts dual binding affinities for both D-Ala-D-Ala and D-Ala-D-Lac and dual antimicrobial activities for both wild type and resistant strains. For example, [ψ[CH₂NH]Tpg⁴]vancomycin aglycon displays a antimicrobial potency that reflects its altered binding characteristics.

### FIELD OF INVENTION

The invention relates to antibacterial antibiotics. More particularly, the invention relates to the reengineering of glycopeptide antibiotics, including vancomycin, to achieve dual D-Ala-D-Ala and D-Ala-D-Lac binding and antibacterial activity with respect to glycopeptide antibiotic resistant bacteria, e.g., VanA resistant bacteria.

### BACKGROUND

The most common strains of enterococci resistant to vancomycin (1), VanA and VanB, possess an inducible resistance pathway in which the terminal dipeptide of the cell wall peptidoglycan precursor is modified from D-Ala-D-Ala to D-Ala-D-Lac (Kahne, D.; et al. Chem. Rev. 2004, 105, 425; Hubbard, B. K.; Walsh, C. T. Angew. Chem. Int. Ed. 2003, 42, 730; Nicolaou, K. C.; et al. Angew. Chem. Int. Ed. 1999, 38, 2096; Williams, D. H.; Bardsley, B. Angew. Chem. Int. Ed. 1999, 38, 1172; Malabarba, A.; et al. Med. Res. Rev. 1997, 17, 69; Glycopeptide resistance and analogues: Malabarba, A.; Ciabatti, R. Curr. Med. Chem. 2001, 8, 1759; Pootoolal, J.; et al. Annu. Rev. Pharmacol. Toxicol. 2002, 42, 381; Van Bambeke, F. V.; et al. Drugs 2004, 64, 913; Sussmuth, R. D. ChemBioChem 2002, 3, 295; Gao, Y. Nat. Prod. Rep. 2002, 19, 100; Healy, V. L.; et al. Chem. Biol. 2000, 7, R109). Binding of the antibiotic to this modified ligand is reduced 1000-fold leading to a 1000-fold drop in antimicrobial activity (Williams, D. H.; Bardsley, B. Angew. Chem. Int. Ed. 1999, 38, 1172; Healy, V. L.; et al. Chem. Biol. 2000, 7, R109). A recent disclosure (McComas, C. C.; et al. J. Am. Chem. Soc. 2003, 125, 9314) disclosed the first experimental study on the origin of this loss in binding affinity, partitioning the effect into lost H-bond and repulsive lone pair contributions, FIG. 1. Thus, the binding affinity of vancomycin for 3, which incorporates a methylene (CH₂) in place of the linking amide NH of Ac₂-L-Lys-D-Ala-D-Ala, was compared with that of Ac₂-L-Lys-D-Ala-D-Ala (2) and AC₂-L-Lys-D-Ala-D-Lac (4). The vancomycin affinity for 3 was approximately 10-fold less than that of 2, but 100-fold greater than that of 4. This indicated that the reduced binding affinity of 4 (4.1 kcal/mol) may be attributed to both the loss of a key H-bond and a destabilizing lone pair/lone pair interaction introduced with the ester oxygen of 4 (2.6 kcal/mol) with the latter, not the H-bond, being responsible for the greater share (100-fold) of the 1000-fold binding reduction. These observations have significant ramifications on the reengineering of vancomycin to bind D-Ala-D-Lac suggesting that the design could focus principally on removing the destabilizing lone pair interaction rather than reintroduction of a H-bond and that this may be sufficient to compensate for two of the three orders of magnitude in binding affinity lost with D-Ala-D-Lac. Thus, synthesis of a vancomycin analogue with removal of the residue 4 carbonyl and its destabilizing lone pair interaction could potentially restore much of the binding affinity of the antibiotic for the modified ligand. At present, such a deep-seated change in the molecule can only be achieved by total synthesis. Efforts to selectively modify the residue 4 carbonyl by selective reaction of the amide linking residues 4 and 5 within vancomycin aglycon derivatives have not yet been successful. Synthetic reviews: Boger, D. L. Med. Res. Rev. 2001, 21, 356; Lloyd-Williams, P.; Giralt, E. Chem. Soc. Rev. 2001, 30, 145; Zhang, A. J.; Burgess, K. Angew. Chem. Int. Ed. 1999, 38, 634; Rao, A. V. R.; et al. Chem. Rev. 1995, 95, 2135; Evans, D. A.; DeVries, K. M. Drugs Pharm. Sci. 1994, 63, 63). Earlier studies have disclosed a convergent synthesis of the vancomycin aglycon (Boger, D. L.; et al. J. Am. Chem. Soc. 1999, 121, 3226; Boger, D. L.; et al. J. Am. Chem. Soc. 1999, 121, 10004) and of the teicoplanin (Boger, D. L.; et al. J. Am. Chem. Soc. 2000, 122, 7416; Boger, D. L.; et al. J. Am. Chem. Soc. 2001, 123, 1862) and ristocetin aglycons (Crowley, B. M.; et al. J. Am. Chem. Soc. 2004, 126, 4310).

What is needed is a reengineered form of glycopeptide antibiotic, including vancomycin, having dual binding affinities with respect to both D-Ala-D-Ala and D-Ala-D-Lac and dual antimicrobial activities with respect to both wild type and glycopeptide antibiotic or VanA resistant organisms. What is needed are compositions and/or processes that employ [ψ[CH₂NH]PG⁴] glycopeptide antibiotic analogs or aglycons wherein the carbonyl of the fourth amino acid residue of the glycopeptide backbone has been replaced with a methylene for imparting dual antimicrobial activities.

### SUMMARY

The first aspect of the invention is directed to a composition having antibacterial activity with respect to glycopeptide antibiotic resistant bacteria and dual binding activity with respect to D-Ala-D-Ala and D-Ala-D-Lac. The composition comprises a [ψ[CH₂NH]PG⁴] glycopeptide antibiotic analog or aglycon combined with a physiologically acceptable carrier. In a preferred embodiment, the [ψ[CH₂NH]PG⁴] glycopeptide antibiotic analog or aglycon is an analog of a glycopeptide antibiotic selected from the group consisting of vancomycins, teicoplanins, balhimycins, actinoidins, ristocetins, and orienticins or of their respective aglycons. Other glycopeptide antibiotics are disclosed by K. C. Nicolaou in Angew. Chem., Int. Ed 1999, 38, 2097. In another preferred embodiment, the [ψ[CH₂NH]PG⁴] glycopeptide antibiotic analog or aglycon is a polycyclic heptapeptide having amino acids numbers 1-7, at least two macrocyclic rings, and an optional sugar unit, wherein amino acids numbers 2, 4 and 6 of said polycyclic heptapeptide each having a side chain containing a benzene ring, amino acid number 4 being a phenyl glycine, each of said macrocyclic rings being independently derived from a bonding together of two different benzene rings of said amino acids, either through an ether linkage or by having the benzene rings being directly bonded together through a sigma bond, the phenyl glycine of amino acid number 4 being bonded at positions 3 and 5 to the benzene rings of the side chains of amino acids number 2 and number 6 through ether linkages or by direct sigma bonding to the benzene ring, and said polycyclic heptapeptide including optional further macrocyclic structures formed between the side chains of amino acids 1 and 3 and/or between the side chains of amino acids 5 and 7 through direct sigma bonds or through ether linkages. In a further preferred embodiment, the [ψ[CH₂NH]PG⁴] glycopeptide antibiotic analog is an aglycon and lacks a sugar unit. In a further preferred embodiment, the [ψ[CH₂NH]PG⁴] glycopeptide antibiotic analog includes at least one sugar unit. In a further preferred embodiment, the [ψ[CH₂NH]PG⁴] glycopeptide antibiotic analog is [ψ[CH₂NH]TPG⁴] vancomycin. In a further preferred embodiment, the [ψ[CH₂NH]PG⁴] glycopeptide antibiotic analog is [ψ[CH₂NH]TPG⁴] vancomycin aglycon.

A second aspect of the invention is directed to a process for decreasing the viability of glycopeptide antibiotic resistant bacteria. In this process, the glycopeptide antibiotic resistant bacteria being of a type that is resistant to either D-Ala-D-Ala or D-Ala-D-Lac binding glycopeptide antibiotics but not both. The process comprises the step of contacting the bacterium with a bactericidal concentration of a [ψ[CH₂NH]PG⁴] glycopeptide antibiotic analog or aglycon, the [ψ[CH₂NH]PG⁴] glycopeptide antibiotic analog or aglycon being of a type having dual binding activity with respect to D-Ala-D-Ala and D-Ala-D-Lac and antibacterial activity with respect to said glycopeptide antibiotic resistant bacteria. In a preferred mode, the [ψ[CH₂NH]PG⁴] glycopeptide antibiotic analog or aglycon is an analog of a glycopeptide antibiotic selected from the group consisting of vancomycins, teicoplanins, balhimycins, actinoidins, ristocetins, and orienticins or of their respective aglycons. In a further preferred mode, the said [ψ[CH₂NH]PG⁴] glycopeptide antibiotic analog or aglycon is a polycyclic heptapeptide having amino acids numbers 1-7, at least two macrocyclic rings, and an optional sugar unit, wherein amino acids numbers 2, 4 and 6 of said polycyclic heptapeptide each having a side chain containing a benzene ring, amino acid number 4 being a phenyl glycine, each of said macrocyclic rings being independently derived from a bonding together of two different benzene rings of said amino acids, either through an ether linkage or by having the benzene rings being directly bonded together through a sigma bond, the phenyl glycine of amino acid number 4 being bonded at positions 3 and 5 to the benzene rings of the side chains of amino acids number 2 and number 6 through ether linkages or by direct sigma bonding to the benzene ring, and said polycyclic heptapeptide including optional further macrocyclic structures formed between the side chains of amino acids 1 and 3 and/or between the side chains of amino acids 5 and 7 through direct sigma bonds or through ether linkages.

A third aspect if the invention is directed to a compound represented by the following structure: 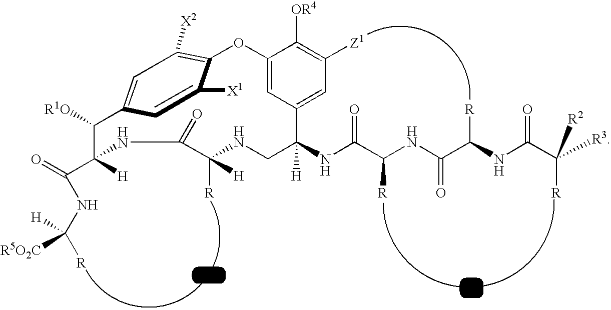
In the above structure, each R is independently selected from the group consisting of amino acid side chains, phenyl rings substituted by one or more chlorines, hydroxy groups, amino groups, sulfates, and sugars; each Z is independently either absent, a sigma bond or a bridging oxygen; Z¹ is a sigma bond or a bridging oxygen; X¹ is either chloro or hydrogen; X² is either chloro or hydrogen; R¹ is selected from the group consisting of hydrogen, sugar, amino sugar, N-alkyl (C1-C6) amino sugar, and acylated amino sugar; R² is hydrogen or with R³ forms a carbonyl group; R³ is selected from the group consisting of amino, methylamino, dimethylamino, and trimethylammonium, or with R² forms a carbonyl group; R⁴ is selected from the group consisting of hydrogen, methyl, sugar, amino sugar, N-alkyl (C1-C6) amino sugar, and acylated amino sugar; and R⁵ is selected from the group consisting of hydrogen, methyl, and C2-C6 alkyl. In a preferred embodiment, the compound is represented by the following structure: 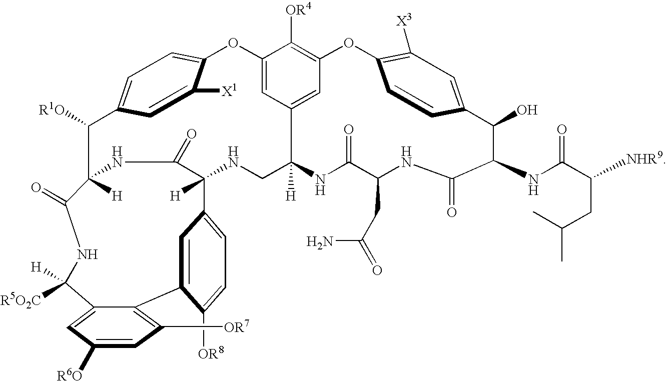
In the above structure, X¹ is either chloro or hydrogen; X³ is either chloro or hydrogen; R¹ is selected from the group consisting of hydrogen, sugar, amino sugar, N-alkylamino sugar, and acylated amino sugar; R⁴ is selected from the group consisting of hydrogen, methyl, sugar, amino sugar, N-alkylamino sugar, and acylated amino sugar; R⁵ is selected from the group consisting of hydrogen, methyl, and C2-C6 alkyl; R⁶ is selected from the group consisting of hydrogen, methyl, sugar, amino sugar, N-alkylamino sugar, and acylated amino sugar; R⁷ is selected from the group consisting of hydrogen, methyl, sugar, amino sugar, N-alkylamino sugar, and acylated amino sugar; R⁸ is selected from the group consisting of hydrogen, methyl, sugar, amino sugar, N-alkylamino sugar, and acylated amino sugar; and R⁹ is hydrogen or methyl. In a further preferred embodiment, the compound is represented by the following structure: 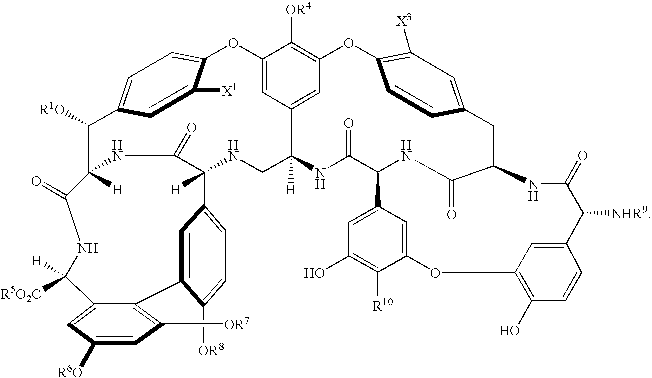
In the above structure, X¹ is either chloro or hydrogen; X³ is either chloro or hydrogen; R¹ is selected from the group consisting of hydrogen, sugar, amino sugar, N-alkyl (C1-C6) amino sugar, and acylated amino sugar; R⁴ is selected from the group consisting of hydrogen, methyl, sugar, amino sugar, N-alkyl (C1-C6) amino sugar, and acylated amino sugar; R⁵ is selected from the group consisting of hydrogen, methyl, and C2-C6 alkyl; R⁶ is selected from the group consisting of hydrogen, methyl, sugar, amino sugar, and acylated amino sugar; R⁷ is selected from the group consisting of hydrogen, methyl, sugar, amino sugar, and acylated amino sugar; R⁸ is selected from the group consisting of hydrogen, methyl, sugar, amino sugar, and acylated amino sugar; R⁹ is hydrogen or methyl; and R¹⁰ is selected from the group consisting of hydrogen, methyl, hydroxyl and amino. In a further preferred embodiment, the compound is represented by the following structure: 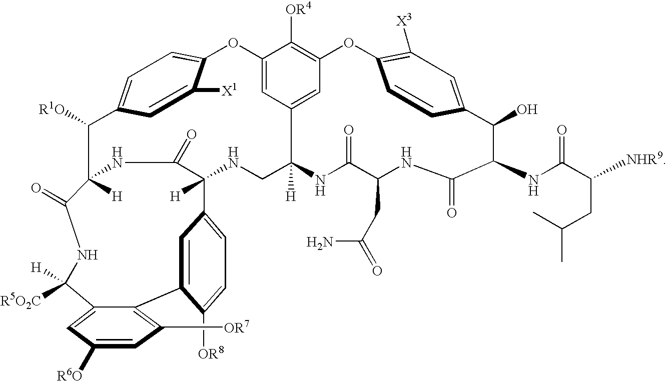
In the above structure, X¹ is either chloro or hydrogen; X³ is either chloro or hydrogen; R¹ is selected from the group consisting of hydrogen and radicals represented by the following structures: 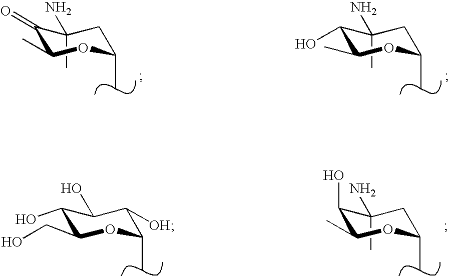
R⁴ is selected from the group consisting of hydrogen, methyl, and radicals represented by the following structures: 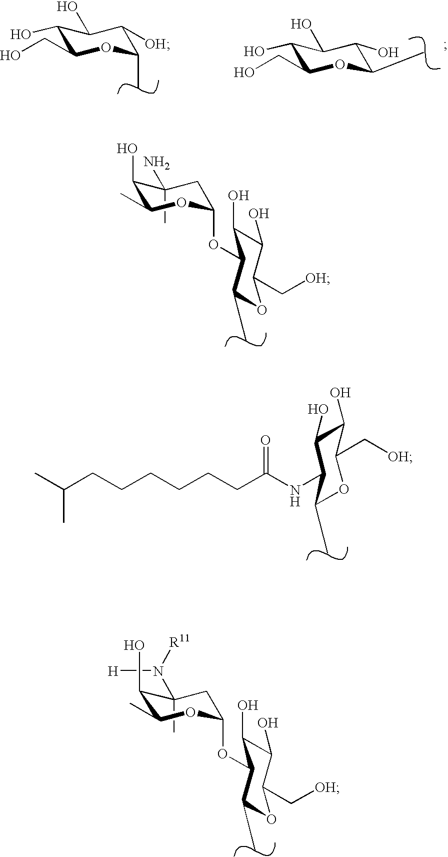
R⁵ is hydrogen or methyl; R⁶ is hydrogen or methyl; R⁷ is hydrogen or methyl; R⁸ is hydrogen or methyl; R⁹ is hydrogen or methyl; R¹¹ is selected from the group consisting of radicals represented by the following structures: 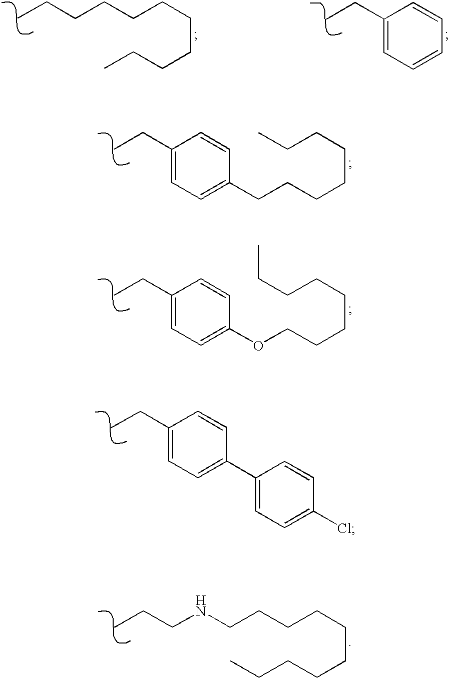
In a further preferred embodiment, the compound is represented by the following structure: 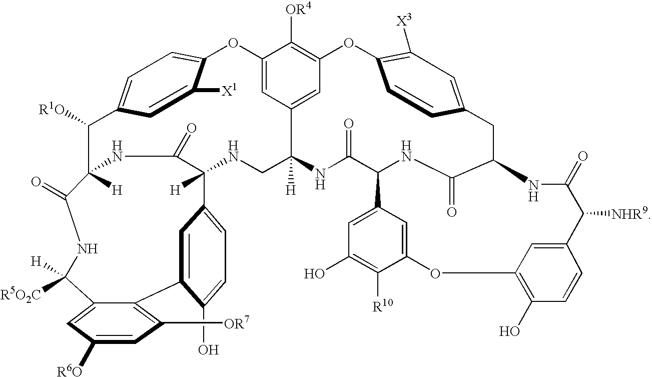
In the above structure, X¹ is either chloro or hydrogen; X³ is either chloro or hydrogen; R¹ is selected from the group consisting of hydrogen, methyl and a radical represented by the following structures: 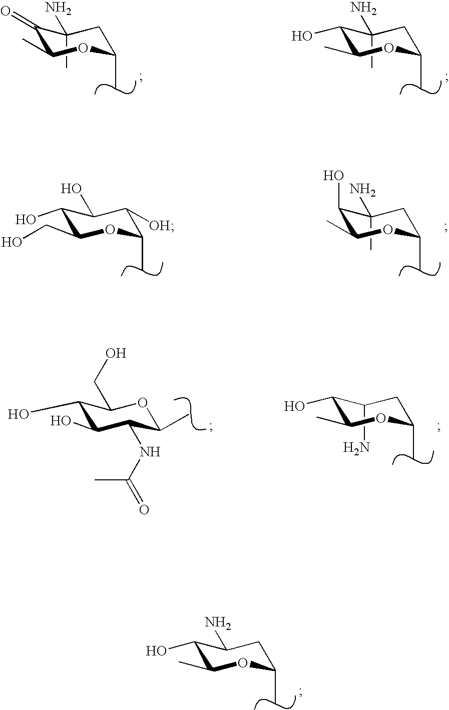
R⁴ is selected from the group consisting of hydrogen, methyl, and a radical represented by the following structures: 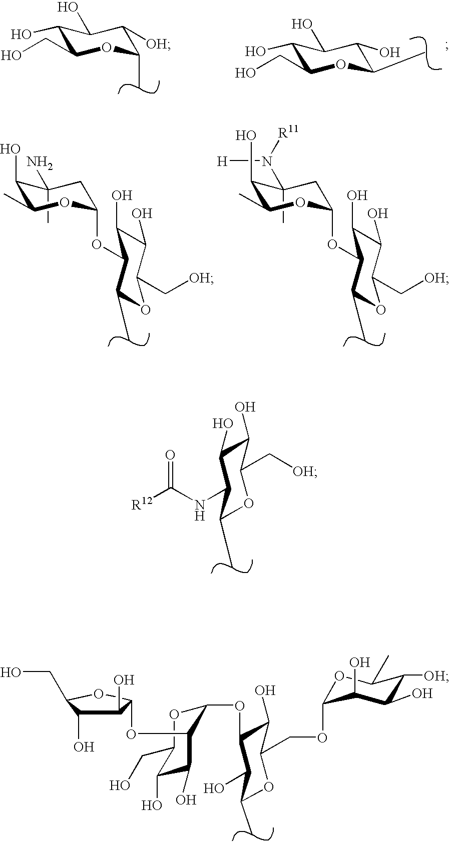
R⁵ is hydrogen or methyl; R⁵ is hydrogen or methyl; R⁷ is selected from the group consisting of hydrogen, methyl and a radical represented by the following structures: 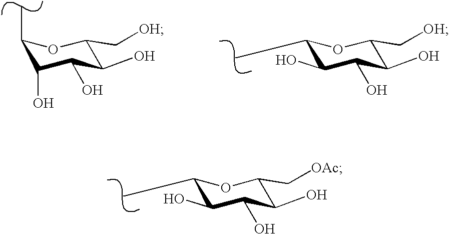
R⁹ is hydrogen or methyl; R¹⁰ is selected from the group consisting of hydrogen, methyl, hydroxyl, and amino; R¹¹ is selected from the group consisting of radicals represented by the following structures: 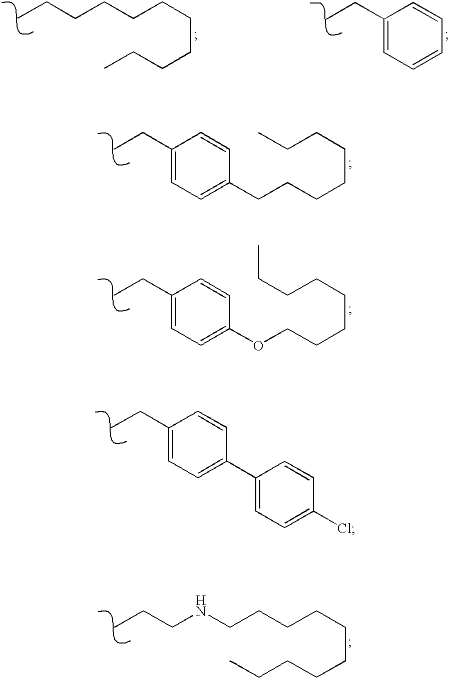
and R¹² is selected from the group consisting of hydrogen, methyl, and radicals represented by the following structures:

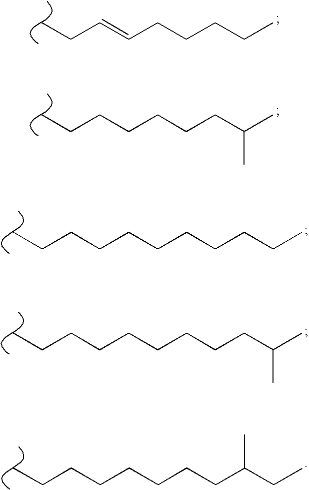

The fourth aspect of the invention is directed to a compound of Formula I represented by the following structure: 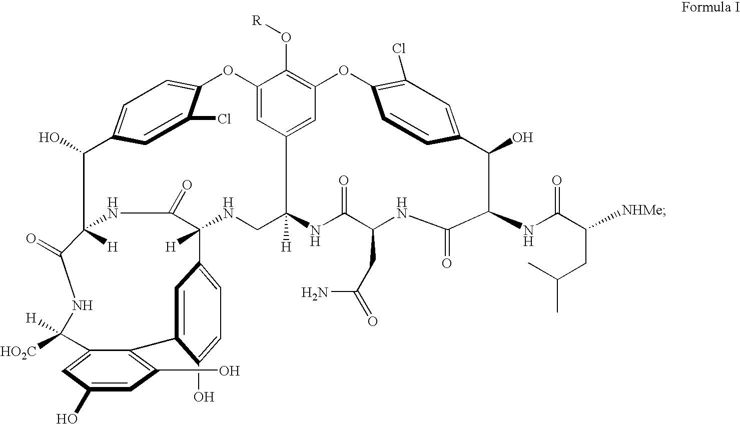
In Formula (I), R is selected from the group of radicals consisting of hydrogen, monosaccharide, disaccharide, and trisaccharide; wherein the mono-, di-, and trisaccharides optionally include one or more amino groups and optionally include one or more (C1-C6) alkyls. In a preferred embodiment, R is a disaccharide represented by the following structure:

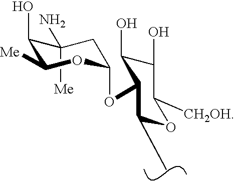

A fifth aspect of the invention is directed to a process for converting compound A into compound B, where A and B are represented by the following structures: 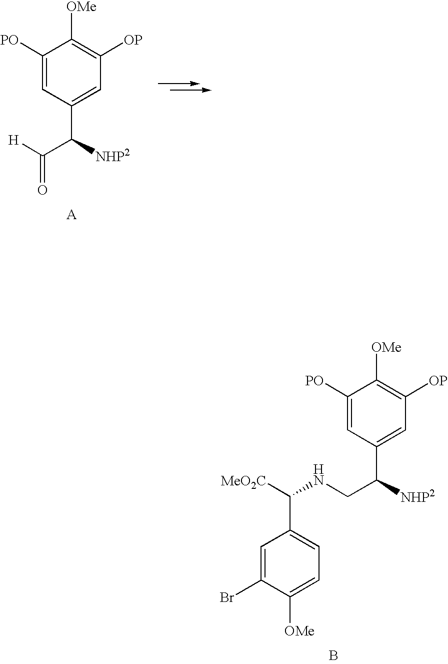
In the first step of the process, compound A is converted to a first intermediate having an imine by reacting the aldehyde of compound A with a second reactant having a primary benzylic amino group for producing the first intermediate. In a preferred mode, the aldehyde of compound A is reacted with a slight excess of the second reactant and in the presence of a dehydrating agent. In the second step, the first intermediate is then converted to compound B. In a preferred mode, the pH of the product of said Step A is adjusted by the addition of glacial acetic acid followed by the addition of a borohydride reagent at a temperature sufficient to allow the reduction of the imine of the first intermediate from step A to be substantially complete after 2 days to give compound B. In Compounds A and B, P and P² are protecting groups. More particularly, P is a protecting group for phenols that can be removed in the presence of phenyl methyl ethers, esters, amines protected by P², phenyl bromides and carbamoyl groups; and P² is a nitrogen protecting group that can be removed in the presence of phenyl chlorides, methyl phenyl ethers, amides, O-MEM groups and benzyl hydroxyl groups.

A sixth aspect of the invention is directed to a process for converting compound B into compound C, wherein compounds B and C are represented by the following structures: 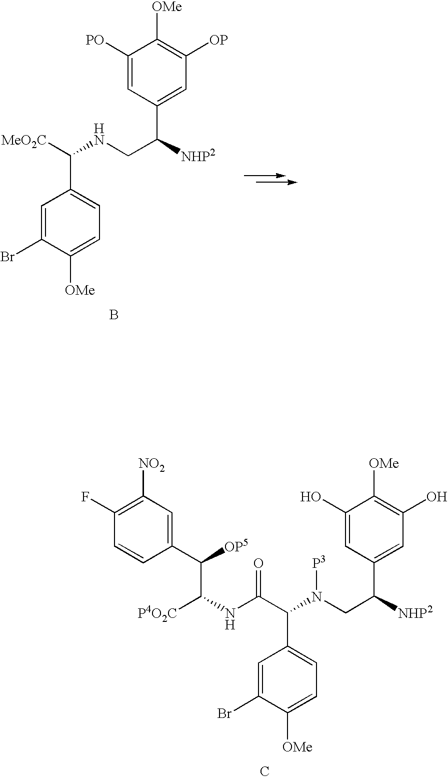
In the first step of the process, compound B is converted to a second intermediate having all protected amino groups, unprotected hydroxyls, and an ester group. In a preferred mode, the free amine of compound B is protected with a protecting group that allows ester hydrolysis, P removal, amide bond formation, Suzuki coupling and diazotization of aniline groups, followed by phenol deprotection by removal of the P protecting groups. In the second step, the second intermediate is then converted to compound C. In a preferred mode, the ester group of the second intermediate is hydrolyzed for revealing a carboxylic acid and forming an amide bond between the carboxylic acid and an ester-protected phenylalanine analog to give compound C. In compounds B and C, P, P², P³, P⁴, and P⁵ are protecting groups. More particularly, P is a protecting group for phenols that can be removed in the presence of phenyl methyl ethers, esters, amines protected by P², phenyl bromides and carbamoyl groups; P² is a nitrogen protecting group that can be removed in the presence of phenyl chlorides, methyl phenyl ethers, amides, O-MEM groups and benzyl hydroxyl groups; P³ is an amine protecting group that is not removed by the reaction conditions for both the first and second steps; P⁴ is an ester protecting group; and P⁵ is a hydroxyl protecting group that is not an ester.

A seventh aspect of the invention is directed to a process for converting compound C into compound D, wherein compounds C and D are represented by the following structures: 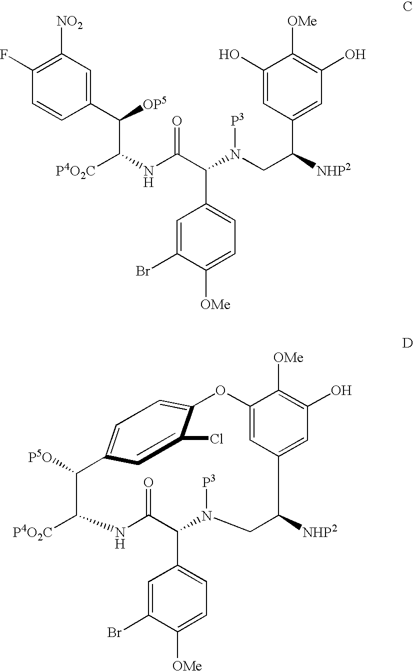
In the first step, compound C is converted to a third intermediate having an aromatic nitro group. In a preferred mode, compound C is converted to the third intermediate by reaction with a suitable base in the presence of a water scavenging agent at a temperature sufficient for macrocyclization to occur by nucleophilic substitution on the nitro group-bearing ring to give a diphenyl ether functionality followed by separating the two resulting atropdiastereomers. In the second step, the third intermediate is then converted to to compound D. In a preferred mode, the third intermediate is converted to compound D by converting the aromatic nitro group to an amine and then reaction with a diazotizing agent and replacement of the diazo group with a chloro group. In compounds C and D, P², P³, P⁴, and P⁵ are protecting groups. More particularly, P² is a nitrogen protecting group that can be removed in the presence of phenyl chlorides, methyl phenyl ethers, amides, O-MEM groups and benzyl hydroxyl groups; P³ is an amine protecting group that is not removed under the reaction conditions of the both first and second steps; P⁴ is an ester protecting group; and P⁵ is a hydroxyl protecting group that is not an ester.

A eighth aspect of the invention is directed to a process for converting compound D and E into compound F, wherein compounds D, E, and F have the following structures: 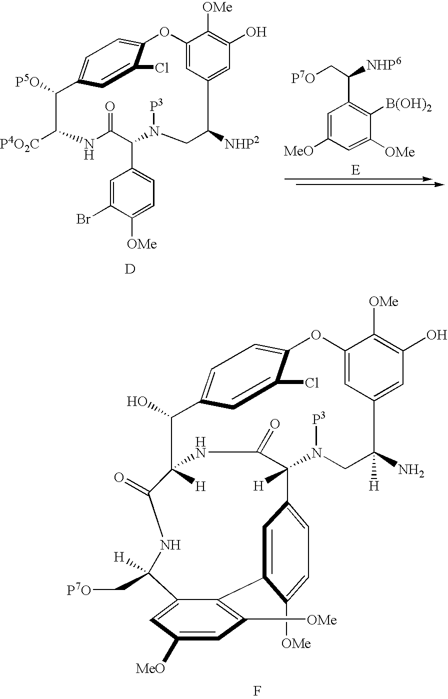
In the first step, compounds D and E are reacted to form a mixture of atropisomers. In a preferred mode, compounds D and E are mixed in the presence of a suitable catalyst to form a mixture of atropisomers whereby the phenyl ring of compound E is bonded to the phenyl ring of compound D at the carbons that formerly were attached to the boron and bromine, respectively, and separating the atropisomers. In the second step, one of the desired atropdiastereomers produced in the first step is then isolated. In a preferred mode, the desired atropdiastereomer is isoloated by heating the undesired atropdiastereomer at a temperature sufficient to convert it to a mixture of atropisomers and again separating the atropisomers; and repeating this second step until a substantial portion of the undesired atropdiastereomer is converted to the desired atropdiastereomer. In the third step, the desired atropdiastereomer of the second step is then deprotected. In a preferred mode, protecting groups P⁵, P⁶ and P⁴ are removed sequentially to give a compound containing a free amino group and a free carboxylic acid. In the fourth step, the deprotected product of the third step is then converted to compound F. In a preferred mode, a dilute solution of the compound of the third step is reacted with a sufficient quantity of amide bond forming reagent to give an intramolecular reaction product; and removal of protecting group P² to afford compound F. In compounds D, E, and F, P², P³, P⁴, P⁵, P⁶, and P⁷ are protecting groups. More particularly, P² is a nitrogen protecting group that can be removed in the presence of phenyl chlorides, methyl phenyl ethers, amides, O-MEM groups and benzyl hydroxyl groups; P³ is an amine protecting group that is not removed by the reaction conditions listed in the first and second steps; P⁴ is an ester protecting group; P⁵ is a hydroxyl protecting group that is not an ester; P⁶ is an amino protecting group; and P⁷ is a hydroxyl protecting group able to be removed in the presence of phenyl methyl ethers and the P³ protecting group.

A ninth aspect of the invention is directed to a process for converting compound F into compound G, wherein the compounds F and G are represented by the following structures: 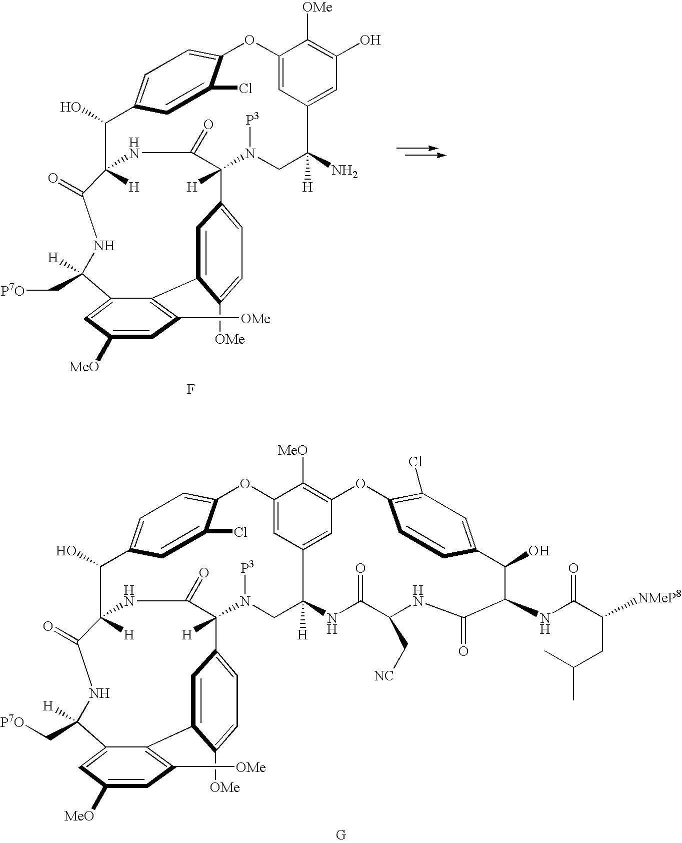
In the first step, compound F is converted to a fourth intermediate having an amide and possessing the full carbon skeleton of the vancomycin analog. In a preferred mode, compound F is reacted with a suitably protected tripeptide free carboxylic acid to give the fourth intermediate. In the second step, the fourth intermediate is converted to a fifth intermediate having a new macrocycle ring possessing a diphenyl ether functionality followed by separation of the desired and undesired atropdiastereomer. In a preferred mode, the fourth intermediate is treated with a suitable fluoride-containing base in the presence of a water scavenging agent to provide a fifth intermediate. In the third step, the fifth intermediate is converted to compound G. In a preferred mode, the aromatic nitro group of the desired atropdiastereomer of the fifth intermediate of said Step B is reduced with a reducing reagent, then the resulting amino group is converted to a diazo group, and then the diazo group is substituted with a chlorine in the presence of a suitable catalyst to give compound G. In compounds F and G, P³, P⁷, and P⁸ are protecting groups. More particularly, P³ is an amine protecting group that is not removed by the reaction conditions listed in the first and second steps; P⁷ is a hydroxyl protecting group able to be removed in the presence of phenyl methyl ethers and the P³ protecting group; and P⁸ is an amino protecting group which is unreactive in the first, second, and third steps.

A tenth aspect of the invention is directed to a process for converting compound G into compound H, wherein compounds G and H are represented by the following structures: 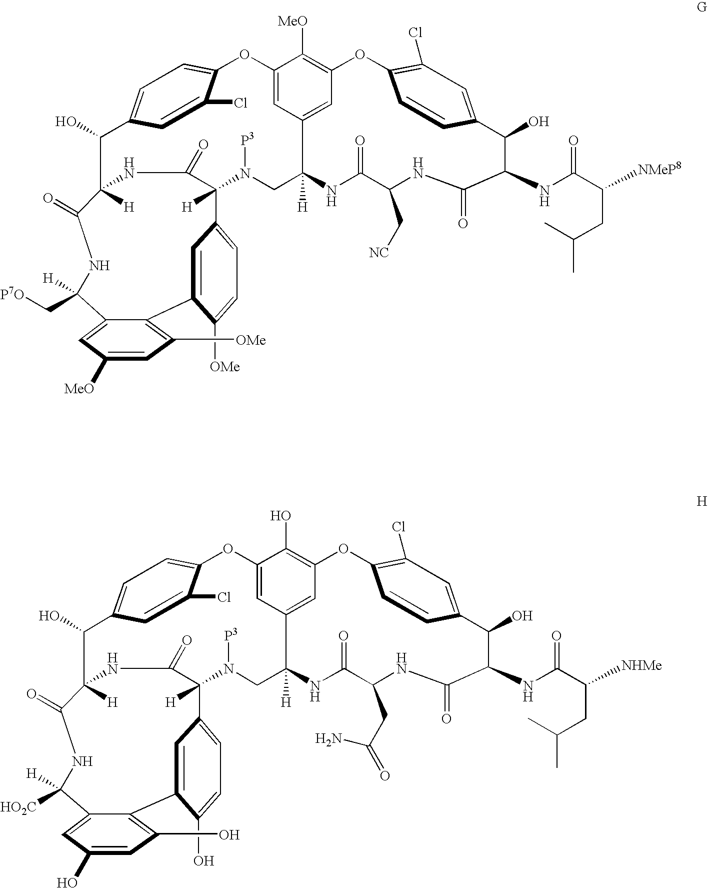
In the first step, compound G is converted to a sixth intermediate having a deprotected hydroxyl at P⁷. In a preferred mode, the benzylic hydroxyl groups of compound G are protected with protecting group P⁹ and the protecting group P⁷ is removed to form the sixth intermediate. In the second step, the sixth intermediate of the first step is then converted to a seventh intermediate having carboxylic acid by oxidizing the primary alcohol of the sixth intermediate to form the carboxylic acid. In a preferred mode, the N-methyl group of the sixth intermediate is reprotected with protecting group P⁸ and the primary alcohol from the resulting compound is oxidized to form the carboxylic acid of the seventh intermediate. In the third step, the seventh intermediate of the second step is then converted to compound H by hydrolyzing the cyano group of the seventh intermediate and removing the remaining protecting groups to give compound H. In a preferred mode, Compound H is formed by hydrolyzing the cyano group of the seventh intermediate of the second step and the remaining protecting groups P³, methyl ethers, P⁸ and P⁹ are removed to give compound H. In compounds G and H, P³, P⁷, P⁸, and P⁹ are protecting groups. More particularly, P³ is an amine protecting group that is not removed by the reaction conditions listed in steps A and B; P⁷ is a hydroxyl protecting group able to be removed in the presence of phenyl methyl ethers and the P³ protecting group; P⁸ is an amino protecting group which is unreactive in said steps A, B and the cyano group hydrolysis of C of claim 7; P⁹ is a hydroxyl protecting group that is not removed under the conditions of steps A and B, and the cyano group hydrolysis of step C.

An effective total synthesis of [ψ[CH₂NH]Tpg⁴]vancomycin aglycon (5) is detailed (26 steps) in which the residue 4 amide carbonyl of the vancomycin aglycon has been replaced with a methylene. This removal of a single atom from the antibiotic was conducted to enhance binding to D-Ala-D-Lac countering resistance endowed to bacteria that remodel their D-Ala-D-Ala peptidoglycan cell wall precursor by a similar single atom change (ester O for amide NH). Key elements of the approach include an effective 14-step synthesis of the modified vancomycin ABCD ring system featuring an early stage reductive amination coupling of residues 4 and 5 for installation of the deep-seated amide modification, the first of two key diaryl ether closures for formation of the modified 16-membered CD ring system (76%, 2.5-3:1 kinetic atropdiastereoselectivity), a remarkably effective Suzuki coupling for installation of the hindered AB biaryl bond (90%) on which the atropisomer stereochemistry could be thermally adjusted, and a final macrolactamization for closure of the 12-membered AB ring system (70%). Subsequent introduction of DE ring system enlisted a room temperature aromatic nucleophilic substitution reaction for formation of the remaining 16-membered diaryl ether (86%, 6-7:1 kinetic atropdiastereoselectivity) completing the carbon skeleton of 5. Consistent with expectations and relative to the vancomycin aglycon, 5 exhibited a 40-fold increase in affinity for D-Ala-D-Lac (Kₐ=5.2×10³ M⁻¹) and a corresponding 35-fold reduction in affinity for D-Ala-D-Ala (Kₐ=4.8×10³ M⁻¹) providing a glycopeptide analogue with balanced, dual binding characteristics. Beautifully, 5 exhibited antimicrobial activity (MIC=31 μg/mL) against a VanA resistant organism (E. faecalis BM4166) that can remodel its D-Ala-D-Ala peptidoglycan cell wall precursor to D-Ala-D-Lac upon glycopeptide antibiotic challenge displaying a potency that reflects these binding characteristics.

### BRIEF DESCRIPTION OF DRAWINGS

FIG. 1 illustrates the factors that determine the binding affinity of Vancomycin and its analogs to the model tripeptide and the rationale for the omission of the carbonyl oxygen of amino acid 4.

FIG. 2 illustrates the retrosynthetic steps used to map out the synthesis of this vancomycin analog. The desired analogue 5 was anticipated to be prepared by a route analogous to that developed for vancomycin (Boger, D. L.; et al. J. Am. Chem. Soc. 1999, 121, 3226; Boger, D. L.; et al. J. Am. Chem. Soc. 1999, 121, 10004), with notable modifications.

FIG. 3 illustrates a scheme showing the synthesis of the BCD “tripeptide.” The B and D subunits 6 and 7 were prepared following previously optimized procedures (see main text for references).

FIG. 4 illustrates a scheme for the synthesis of the ABCD ring system starting from N-Boc amino ester diamide 14.

FIG. 5A illustrates a table summarizing the conditions tested for the cyclization of 14 to 15.

FIG. 5B illustrates a table summarizing the conditions used for the cyclization of 14 to 15 after conditions in FIG. 5A were tried.

FIG. 6 illustrates a short scheme showing the steps taken to attempt to recycle the undesired atropdiastereomers 15 and 17 by heating in solvent and how they were identified as atropisomers of 16 and 18, respectively.

FIG. 7 illustrates the synthesis of the complete carbon skeleton of the vancomycin aglycon analog.

FIG. 8 illustrates a table that shows the conditions used for the cyclization of 29 to form 30 by catalyzing with a fluoride ion in the presence of added base.

FIG. 9 illustrates a drawing showing the different modifications in the vancomycin structure of analogs that are possible and what relative affinity they have for either the D-Ala-D-Ala ligand or the D-Ala-D-Lac ligand.

FIG. 10 illustrates an N-Boc deprotection of 33 to give 41 without deprotecting the methyl carbamate of residue 4 and removing the MEM group. Compound 41 was synthesized to test its binding affinity in comparison with vancomycin, 5 and 38.

FIG. 11 illustrates a table showing the results of the assessment of 5 alongside vancomycin (1) and its aglycon 38 and structure 41.

FIG. 12 illustrates the structure of the vancomycin analog and its binding constant with the two model ligands.

FIG. 13 illustrates a Skatchard analysis of compound 5 with the N,N′-Ac₂-Lys-D-Ala-D-Ala ligand.

FIG. 14 illustrates a Skatchard analysis of compound 5 with the N, N′-Ac₂-Lys-D-Ala-D-Lac ligand.

FIG. 15 illustrates a titration curve of 5 and the N,N′-Ac₂-Lys-D-Ala-D-Ala ligand.

FIG. 16 illustrates a titration curve of 5 and the N, N′-Ac₂-Lys-D-Ala-D-Lac ligand.

FIG. 17 illustrates important modifications to the basic vancomycin analog structure.

### DEFINITIONS

Unless other qualified herein, the term “glycopeptide antibiotic” is defined herein as a polycyclic heptapeptide containing at least one sugar unit and containing at least two macrocyclic rings. The cyclic structures are derived from the bonding together of two different aromatic side chains of the amino acids, either through an ether linkage or by having the aromatic rings directly bonded together through a sigma bond. The fourth amino acid, a phenyl glycine, is bonded to the side chains of amino acids number 2 and number 6 at positions 3 and 5 on its phenyl ring through ether linkages or by directly bonding to the aromatic ring. The fourth amino acid, i.e., the phenyl glycine, is also sometimes known as the central amino acid. Additional macrocyclic structures, if any, are formed between the side chains of amino acids 1 and 3 and between the side chains of amino acids 5 and 7 through direct sigma bonds or through ether linkages.

Unless other qualified herein, the term “glycopeptide antibiotic aglycone” is defined herein as a glycopeptide antibiotic as defined previously, vide supra, except that no sugar moiety is attached to it.

Unless other qualified herein, the term “[ψ[CH₂NH]PG⁴] glycopeptide antibiotic analog” is defined herein as a glycopeptide antibiotic as defined previously, vide supra, except that the carbonyl of the fourth amino acid residue, i.e, the phenyl glycine, is replaced by a methylene group. The introduction of this methylene group results in the replacement of the peptide linkage between the fourth and fifth amino acid residues with a sigma bond.

Unless other qualified herein, the term “[ψ[CH₂NH]PG⁴] glycopeptide antibiotic analog aglycone” is defined herein as a glycopeptide antibiotic analog as defined previously, vide supra, except that no sugar moiety is attached to it.

Unless other qualified herein, the term “sugar” is defined as a mono-, di-, tri- or tetrasaccharide unit that may or may not be branched or linear made up of saccharide units containing between 5 and 7 carbon atoms in a 5- or 6-membered heterocyclic ring having a single oxygen atom as the heteroatom.

Unless other qualified herein, the term “amino sugar” is defined as a mono-, di-, tri- or tetrasaccharide unit that may or may not be branched or linear made up of saccharide units containing between 5 and 7 carbon atoms in a 5- or 6-membered heterocyclic ring having a single oxygen atom as the heteroatom an containing at least one amino group bonded to only one carbon atom through a sigma bond.

Unless other qualified herein, the term “N-alkylated amino sugar” is defined as an amino sugar having an additional alkyl group on the amino group of the amino sugar. The amino group is disubstituted. The alkyl groups attached to the nitrogen may be simple alkyl groups or the alkyl groups may contain double bonds or one or more aromatic rings that may be additionally substituted with heteroatoms or alkyl groups.

Unless other qualified herein, the term “N-acylated amino sugar” is defined as an amino sugar that has the amino group attached to an acyl group through an amide bond. The amino group is disubstituted. The acyl group may be contain simple alkyl groups or it may contain double bonds or aromatic rings that may be additionally substituted.

### DETAILED DESCRIPTION

The modification of the dipeptide terminus of peptidoglycan cell wall precursors from D-Ala-D-Ala to D-Ala-D-Lac in resistant bacteria reduces the binding affinity of vancomycin for the ligand by 1000-fold leading to a 1000-fold loss in biological activity. It had earlier been shown that a modified peptide ligand possessing a methylene in place of the lactate oxygen restores 100-fold of this binding affinity by removal of a destabilizing lone pair interaction. It is disclosed herein that removal of the residue 4 carbonyl in the vancomycin aglycon produces an analogue with enhanced affinity for D-Ala-D-Lac and restores much of the biological activity of the molecule that is lost with resistant bacteria. Moreover and among the range of potential modifications that could be envisioned, that entailing the simple removal of the residue 4 carbonyl providing 5 are disclosed to bind D-Ala-D-Ala and D-Ala-D-Lac with similar affinities providing an analogue having equivalent effectiveness against sensitive (D-Ala-D-Ala) and resistant (D-Ala-D-Lac) bacteria. Efforts were extended on the preparation of glycopeptide antibiotics to a total synthesis of the [ψ[CH₂NH]Tpg⁴]vancomycin aglycon (5) in which the residue 4 carbonyl has been replaced with a methylene. Consistent with expectations and relative to the vancomycin aglycon, 5 exhibited a 40-fold increase in affinity for D-Ala-D-Lac (Kₐ=5.2×10³ M⁻¹) and a corresponding 35-fold reduction in affinity for D-Ala-D-Ala (Kₐ=4.8×10³ M⁻¹) providing a molecule with balanced, dual binding characteristics. Compound 5 exhibited antimicrobial activity against a VanA resistant organism that remodels its D-Ala-D-Ala peptidoglycan cell wall precursor to D-Ala-D-Lac upon glycopeptide challenge displaying a potency that reflects these binding characteristics.

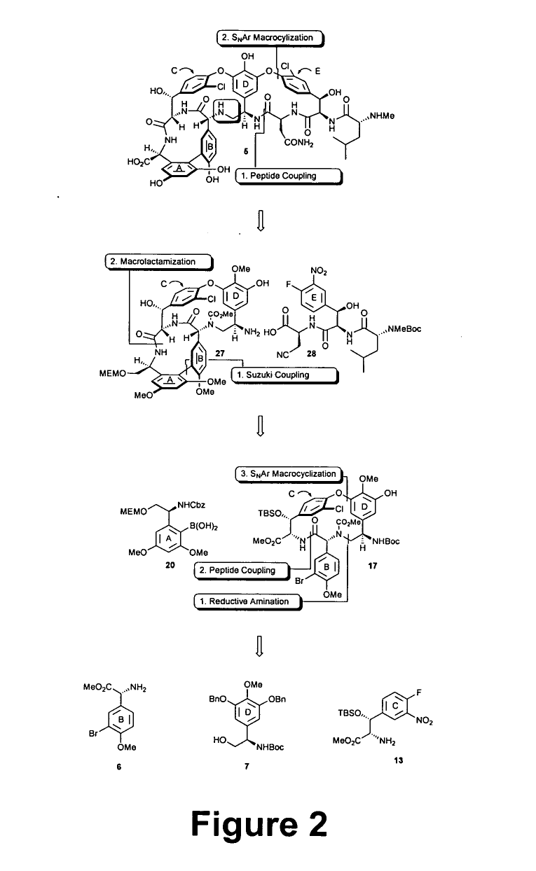

Challenges and Synthetic Plan for [ψ[CH₂NH]Tpg⁴]Vancomycin Aglycon. The desired analogue 5 was anticipated to be prepared by a route analogous to that developed for vancomycin (Boger, D. L.; et al. J. Am. Chem. Soc. 1999, 121, 3226; Boger, D. L.; et al. J. Am. Chem. Soc. 1999, 121, 10004), with notable modifications. Thus, two aromatic nucleophilic substitution reactions with formation of the biaryl ethers are enlisted for CD and DE macrocyclization, a key macrolactamization reaction are employed for cyclization of the AB ring system, and the defined order of CD, AB, and DE ring closures permit sequential control of the atropisomer stereochemistry of each of the newly formed ring systems or their immediate precursors, FIG. 2. Thus, in addition to any kinetic diastereoselection that may be achieved in the ring closures, this order is disclosed to permit the recycling of any undesired atropisomer for each newly introduced ring system by thermal equilibration providing a predictable control of the stereochemistry and dependably funneling all synthetic material into one of eight possible atropdiastereomers. Key to recognition of this preferential order of ring closures was the establishment of the thermodynamic parameters of atropisomerism for the individual vancomycin ring systems: DE ring system (Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920) (Eₐ=18.7 kcal/mol)<AB biaryl precursor (Boger, D. L.; et al. J. Am. Chem. Soc. 1999, 121, 3226; Boger, D. L.; et al. J. Am. Chem. Soc. 1999, 121, 10004) (Eₐ=25.1 kcal/mol)<CD ring system (Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920) (Eₐ=30.4 kcal/mol). Thus, the molecule was assembled by coupling the modified and fully functionalized ABCD ring system 27 with the E ring tripeptide 28 followed by a diastereoselective aromatic nucleophilic substitution reaction for closure of the 16-membered DE ring system with formation of the biaryl ether linkage. Notably, the activating nitro substituent additionally serves as the precursor functionality for aryl chloride introduction and the analogous vancomycin ring closures (Boger, D. L.; et al. J. Am. Chem. Soc. 1999, 121, 3226; Boger, D. L.; et al. J. Am. Chem. Soc. 1999, 121, 10004; Boger, D. L.; et al. Bioorg. Med. Chem. Lett. 1995, 5, 3091; Evans, D. A.; Watson, P. S. Tetrahedron Lett. 1996, 37, 3251; Evans, D. A.; et al. Angew. Chem., Int. Ed. 1998, 37, 2700; Evans, D. A.; et al. Angew. Chem,. Int. Ed. 1998, 37, 2704) were effected with preferential formation of the natural (P)-atropisomer. The E ring tripeptide 28 was derived in the manner described for vancomycin (Boger, D. L.; et al. J. Am. Chem. Soc. 1999, 121, 3226; Boger, D. L.; et al. J. Am. Chem. Soc. 1999, 121, 10004) except that the E ring subunit was prepared enlisting an improved route developed during a more recent total synthesis of the ristocetin aglycon (Crowley, B. M.; et al. J. Am. Chem. Soc. 2004, 126, 4310) employing α-hydroxypinanone (Solladié-Cavallo, A.; Nsenda, T. Tetrahedron Lett. 1998, 39, 2191) as the chiral auxiliary for a diastereoselective aldol addition. The most significant deviations rest with the required modifications in the preparation of the ABCD subunit which house the modified amide and include the use of a reductive amination coupling of residues 4 and 5 (D and B rings) with protection of the newly generated amine as a methyl carbamate and an experimentally-derived altered order to the assembly of the BCD tripeptide. A relatively small and robust amine protecting group was chosen to avoid the introduction of unfavorable steric interactions that affects the CD macrocyclic ring closure and that is stable throughout the synthesis, yet still compatible with a final stage global deprotection. CD macrocyclization enlisting a key aromatic nucleophilic substitution reaction for formation of 16-membered biaryl ether followed by Suzuki coupling of the A ring subunit and AB macrolactamization was employed to complete the preparation of the modified ABCD ring system 27 enlisting a ring closure order that permits the sequential and selective thermal adjustment of the CD and AB ring system atropisomer stereochemistry. Key unforeknown features of the approach include the feasibility of conducting the critical CD ring closure enlisting the residue 4 protected amine versus amide, the resulting unknown atropisomer stereochemical issues (kinetic and thermodynamic diastereoselectivity), and the impact the deep-seated structural change on the conformational features of the CD or ABCD ring systems and those of the final molecule. Finally, the subtle choices of a nitrile as a precursor to the residue 3 side chain carboxamide permits a final stage amide deprotection yet conveys stability throughout the synthesis to any projected thermal atropisomer equilibrations in its presence (Boger, D. L.; et al. J. Am. Chem. Soc. 1998, 120, 8920), and the use of a MEM protected hydroxymethyl precursor (vs a methyl ester) to the C-terminus carboxylic acid enhances the rate of the projected AB macrolactamization (Boger, D. L.; et al. J. Am. Chem. Soc. 1999, 121, 3226; Boger, D. L.; et al. J. Am. Chem. Soc. 1999, 121, 10004) and precludes inadvertent epimerization throughout the synthesis.

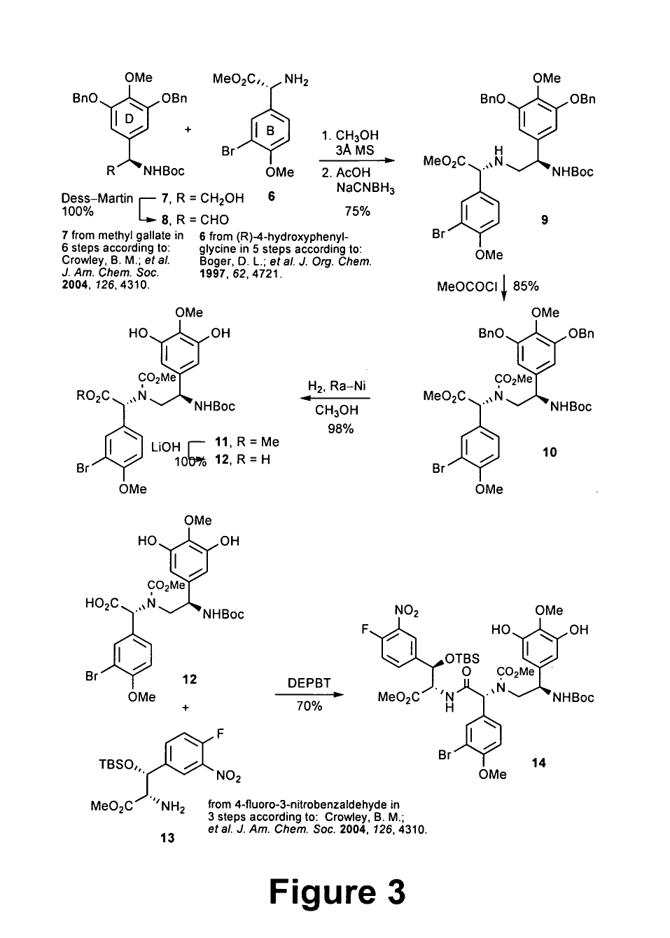

Synthesis of the BCD Tripeptide. The B and D subunits 6 and 7 were prepared following previously optimized procedures (Crowley, B. M.; et al. J. Am. Chem. Soc. 2004, 126, 4310; Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920). Oxidation of alcohol 7 (Compound 7 is available in 6 steps (37% overall) from methyl gallate using 3 recrystallizations and was scaled to 300 g, (Crowley, B. M.; et al. J. Am. Chem. Soc. 2004, 126, 4310)) (2.0 equiv of Dess-Martin periodinane, CH₂Cl₂, 0-25° C., 1 h, 100%) was followed by immediate reductive amination coupling of the sensitive aldehyde 8 with 6 (Compound 6 is available in 5 steps (55% overall) from (R)-4-hydroxyphenyl-glycine using 2 recrystallizations and was scaled to 60 g, (Boger, D. L.; et al. J. Org. Chem. 1997, 62, 4721)) (1.1 equiv, CH₃OH, 3 Å MS, 0° C., 45 min; 3.0 equiv of AcOH, 3.0 equiv of NaBH₃CN, −20° C., 2 d) to afford amine 9 in good yield (75%) and excellent diastereoselectivity (12:1), FIG. 3. Shorter reaction times (14-20 h) at higher temperatures (−15 to −5° C.) led to substandard selectivities (4:1 to 9:1) and the use of less NaBH₃CN (1.5-2.0 equiv) at lower temperatures (−20° C.) led to incomplete reactions. Longer reaction times (3-8 d) led to only marginal increases in yield (82% after 8 d) and roughly equal diastereoselectivities. Initial efforts to prepare amine 9 directly by displacement of the mesylate derived from alcohol 7 were ineffective as were attempts to conduct the reductive amination with the BC dipeptide and 8. Amine protection of 9 as the methyl carbamate (10 equiv of MeOCOCl, 10 equiv of K₂CO₃, THF, 0-25° C., 18 h, 85%) followed by benzyl ether deprotection (Benzyl ether deprotection at higher temperatures (25° C.) may lead to competitive aryl bromide reduction although this was only observed in appreciable amounts when excess Raney Ni was employed.) (Raney Ni, CH₃OH, 0° C., 5 h, 98%) and saponification (3.0 equiv of LiOH, THF—H₂O, 0° C., 6 h, 100%) provided 12. Unexpectedly, the order of these latter two deprotections proved important. Saponification of 10 (Saponification of 11 was considerably slower than that of 10 and occasionally required additional LiOH for complete conversion to 12 with little effect on the amount of epimer generated in the reaction.) under a variety of conditions (LiOH, THF—H₂O or t-BuOH—H₂O, −10 to 0° C.; LiOOH, THF—H₂O; Me₃SnOH, 1,2-dichloroethane, 70° C.) led to variable amounts of an epimer (5-20%) that was difficult to separate from the product. In contrast, benzyl ether deprotection of 10 followed by saponification of 11 reduced the amount of epimer (0-3%) presumably due to preferential deprotonation of the phenols such that subsequent C_{α} deprotonation at residue 5 was less facile (Saponification of 11 was considerably slower than that of 10 and occasionally required additional LiOH for complete conversion to 12 with little effect on the amount of epimer generated in the reaction.). Coupling of 12 with 13 (Compound 13 is available in 3 steps (45% overall) from 4-fluoro-3-nitrobenzaldehyde and was scaled to 30 g, (Crowley, B. M.; et al. J. Am. Chem. Soc. 2004, 126, 4310).) (3.0 equiv of DEPBT (Fan, C.-X.; et al. Org. Lett. 1999, 1, 91), 3.0 equiv of NaHCO₃, DMF, 0-25° C., 8 h) gave tripeptide 14 in good yield (70%) and excellent diastereoselectivity (14:1). A range of other more conventional coupling reagents (EDCl—HOAt, HATU, FDPP) also provided good conversions (65-80%), but suffered from considerable competitive racemization.

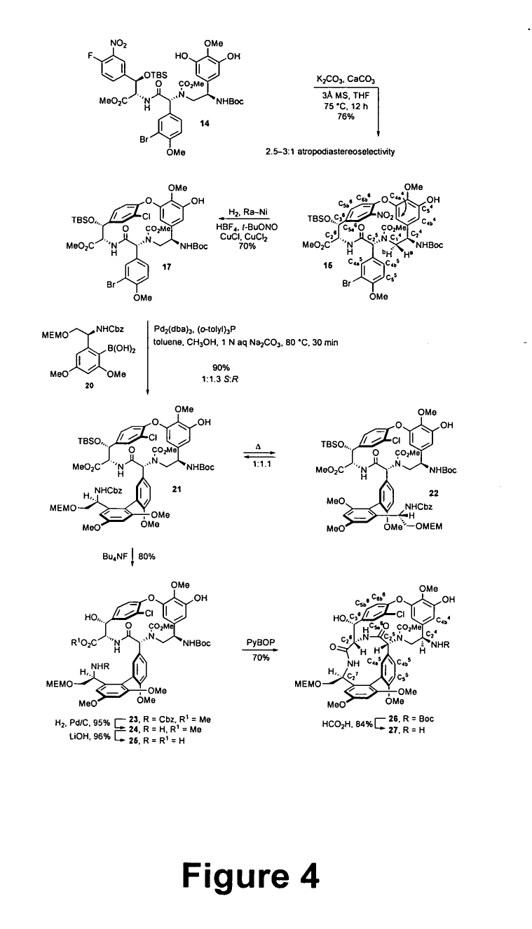

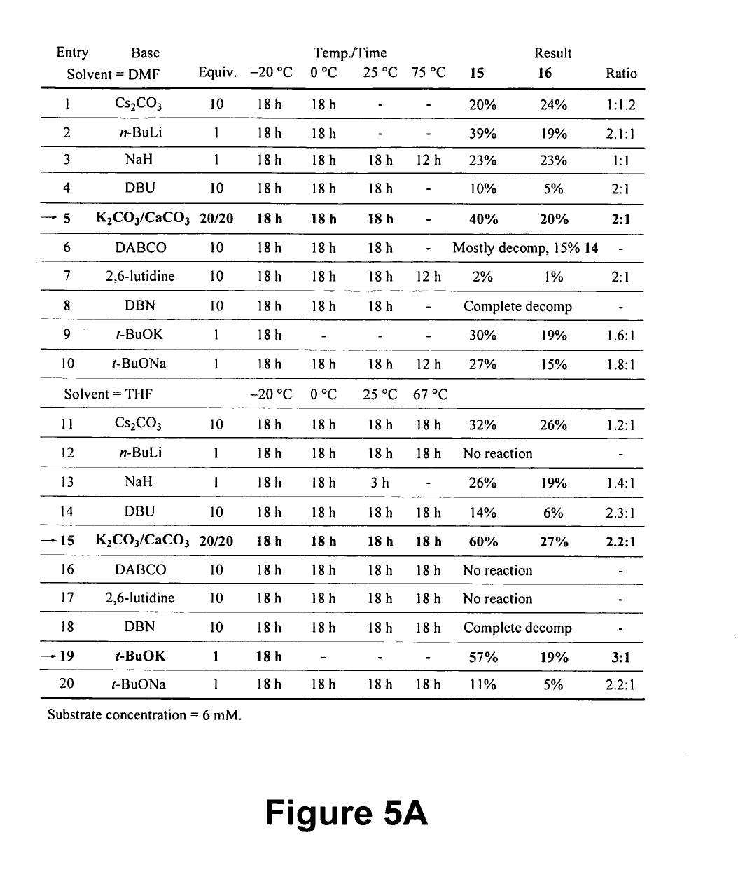

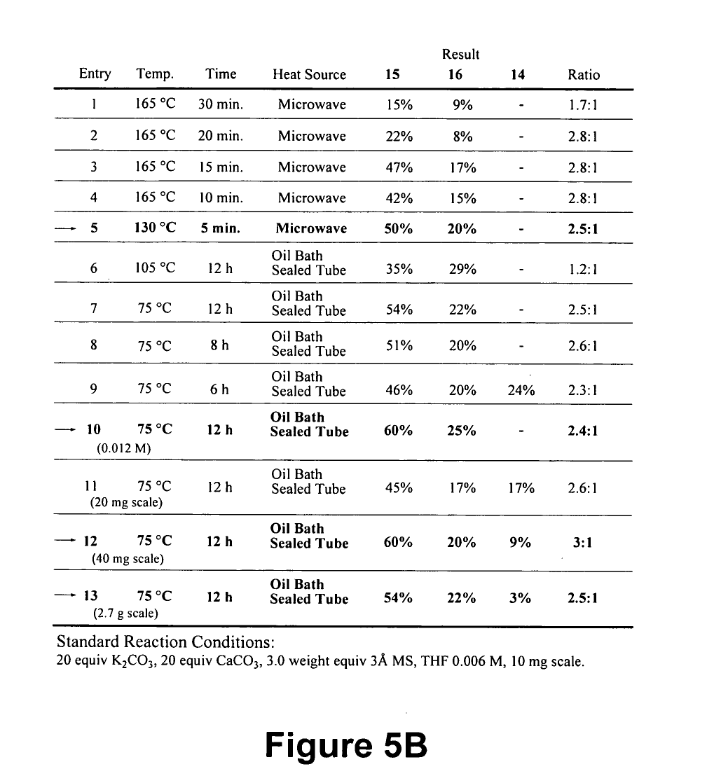

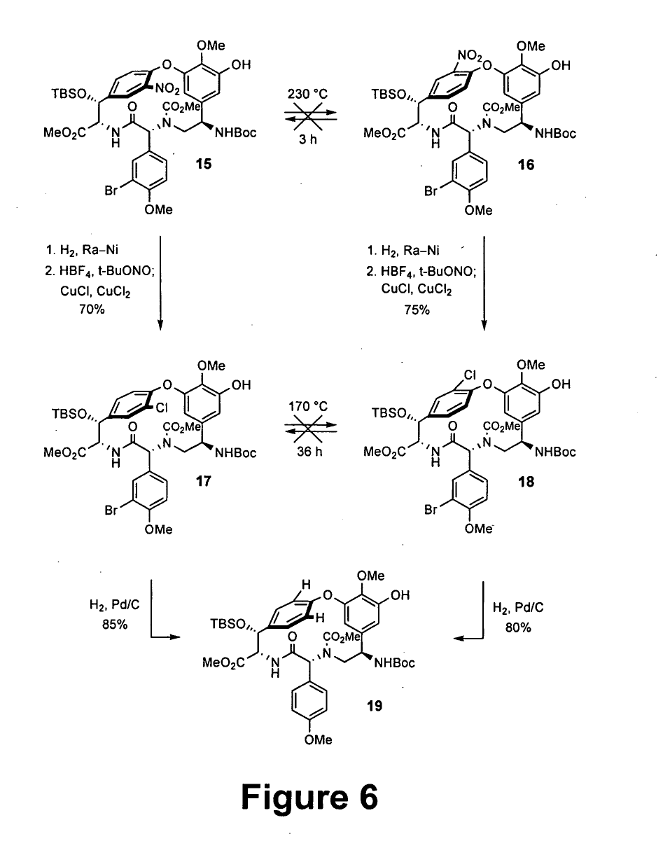

Synthesis of the ABCD Ring System. This set the stage for a detailed examination of one of the critical reactions in the approach to 5 entailing the cyclization of 14. After considerable optimization (FIGS. 5A and 5B), cyclization of 14 (20 equiv of K₂CO₃, 20 equiv of CaCO₃, 3 wt equiv of 3 Å MS, 12 mM THF, 75° C. bath temp, 12 h) afforded 15 in good yield (54%) and good atropodiastereoselectivity (2.5:1, 15 (54%) and 16 (22%)) even when conducted on a large scale (2.7 g), FIG. 4. The inclusion of CaCO₃ in the reaction mixture is critical and serves to trap the liberated fluoride arising from the aromatic nucleophilic substitution as an insoluble byproduct (CaF₂) preventing TBS ether deprotection and a subsequent competitive base-catalyzed retro aldol reaction of the free alcohol. Nearly comparable results were obtained by promoting the ring closure of 15 with the stronger base t-BuOK (1.0 equiv, THF, −20° C., 18 h) providing 15 and its atropisomer 16 in 57% and 19% (3:1 atropodiastereoselectivity), respectively, under remarkably mild reaction conditions (−20° C., THF). However, the use of t-BuOK proved more sensitive to the reaction parameters, suffered competitive racemization if excess base was employed, and proved more difficult to implement on a large scale than the reaction enlisting K₂CO₃/CaCO₃. The cyclization of 14 represents a considerable improvement over the analogous ring closure reaction enlisted in an earlier synthesis of vancomycin (50-65%, 1:1 atropisomers vs 76-87%, 2.5-3:1 atropisomers) where both the overall conversion and atropodiastereoselectivity were lower illustrating that the closure of 14 may benefit from both the increased conformational flexibility of the cyclization substrate and the residue 4 amine small protecting group. Unlike the vancomycin CD ring system in which the atropisomers could be thermally equilibrated at 120-140° C. permitting the recycling and productive use of the unnatural atropisomer, the atropisomers 15 and 16 could not be thermally interconverted even at temperatures as high as 210-230° C., FIG. 6.

Reduction of the nitro group (Raney Ni, 0° C., CH₃OH, 1 h) followed by diazotization (1.3 equiv of HBF₄, 1.3 equiv of t-BuONO, CH₃CN, 0° C., 30 min) and Sandmeyer substitution (50 equiv of CuCl, 60 equiv of CuCl₂, H₂O, 0-25° C., 1 h, 70% from 15) cleanly provided 17 without loss of the atropisomer stereochemistry inherent in starting 15. The unnatural atropisomer 16 was also subjected to these conditions to cleanly give 18 (75%) (FIG. 6). The stereochemical assignments of these two compounds and their relationship as atropisomers (vs epimers) were established by 2D ROESY ¹H—¹H NMR experiments and confirmed chemically by their reductive dechlorination (H₂, 10%, Pd/C) to afford the identical product 19 (FIG. 6).

Suzuki coupling of 17 with the hindered A ring boronic acid 20 (Boger, D. L.; et al. J. Am. Chem. Soc. 1999, 121, 3226; Boger, D. L.; et al. J. Am. Chem. Soc. 1999, 121, 10004) (0.3 equiv of Pd₂(dba)₃, 1.5 equiv of (o-tol)₃P, toluene-CH₃OH-1 N aq Na₂CO₃ 10:3:1, 80° C., 30 min) proceeded in excellent yield (90%) under remarkably effective conditions (Boger, D. L.; et al. J. Am. Chem. Soc. 1999, 121, 3226; Boger, D. L.; et al. J. Am. Chem. Soc. 1999, 121, 10004) given the steric constraints of the substrate 20 providing a separable 1:1.3 mixture of atropisomers (21:22) slightly favoring the unnatural configuration. Thermal equilibration of isolated 22 was carried out initially employing reported conditions for vancomycin (o-dichlorobenzene, 120° C., 18 h, 81% recovery of material) (Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920) to afford a 1:1.1 separable mixture permitting the recycling of this unnatural atropisomer. An examination of the parameters for this isomerization (k=0.12 h⁻¹, t_{1/2}=5.9 h at 120° C. and k=0.36 h⁻¹, t_{1/2}=1.8 h at 135° C.) revealed that it proceeds with an energy of activation (Eₐ) of 25.6 kcal/mol (ΔH^{‡}=24.8 kcal/mol, ΔS^{‡}=−0.26 e.u., ΔG^{‡}=24.9 kcal/mol) essentially indistinguishable from that observed with the authentic vancomycin AB biaryl system, but it does not result in the analogues 3:1 thermodynamic preference for the natural atropisomer. However, the unusual and unexpected atropisomer stability of the CD ring system allowed us to improve on the recycling conditions. Heating the mixture in a microwave reactor at an elevated temperature (210° C., o-dichlorobenzene) shortened the reaction time significantly (5 min vs 18 h) and slightly improved the recovery of material (88% vs 81%). This improvement impacted the efficiency of the recycling of 22 by allowing multiple equilibrations to be run in a single day rather than over the course of a week. Silyl ether deprotection of 21 (1.2 equiv of Bu₄NF, THF, 0° C., 10 min) followed by N-Cbz removal (H₂, 10% Pd/C, 1% Cl₃CCO₂H—CH₃OH, 15 min, 95%) and methyl ester hydrolysis (1.0 equiv of LiOH, THF—H₂O, 0° C., 1 h, 96%) gave amino acid 25. Notably, N-Cbz removal in the absence of Cl₃CCO₂H (Boger, D. L.; et al. J. Am. Chem. Soc. 2000, 122, 7416; Boger, D. L.; et al. J. Am. Chem. Soc. 2001, 123, 1862) was much slower (11 h) and these conditions led to competitive chloride reduction (Use of Raney Ni for N-Cbz removal was also successful, though lower recoveries (84%) of the product were observed.). Macrolactamization with closure of the AB ring system was effected by treatment of 25 with PyBOP (3.0 equiv, 6.0 equiv of NaHCO₃, 0.001 M CH₂Cl₂-DMF 5:1, 0-25° C., 12 h) to afford the fully functionalized bicyclic ABCD ring system 26 in good yield (70%) with only trace amounts of competitive epimerization (<3%). Alternative coupling reagents (EDCl and HOAt or HOBt, HATU) and reaction conditions (10-100% DMF—CH₂Cl₂, 3-5 equiv of Na₂CO₃, −5 to 0° ) led to lower conversions (30-52%) or required extended reaction times (3 d). N-Boc deprotection (HCO₂H—CHCl₃ 1:1, 10 h, 84%) gave the free amine 27 for coupling with the E ring tripeptide. Confirmation of the atropisomer stereochemistry and amide conformational assignments for 26 were established by 2D ROESY ¹H—¹H NMR. Diagnostic NOE crosspeaks for 26 were observed between C₅⁴—OH/C_{4b}⁴—H (s), C₅⁴—OH/C₆⁴—OMe (s), N₁⁷—H/C₄ₐ⁵—H (s), N₁⁷—H/C₂⁵—H (s), N₁⁷—H/C₃⁶—H (m), N₁⁷—H/C₂⁶—H (m), C₅ₐ⁶—H/C₃⁶—H (s), C₅ₐ⁶—H/C₂⁶—H (s), C_{5b}⁶—H/N₁⁶—H (m), C₃⁶—OH/N₁⁶—H (s), C_{5b}⁶—H/C₃⁶—OH (m), C_{6b}⁶—H/C_{5b}⁶—H (s), C_{6b}⁶—H/C₄ₐ⁴—H (w), N₁⁴—H/C_{4b}⁴—H (m), N₁⁴—H/C₄ₐ⁴—H (w), C_{4b}⁵—H/C₅⁵—H (s), C₂⁶—H/C₄ₐ⁵—H, C_{4b}⁵—H/C_{1b}⁴—H (m), C₄ₐ⁵—H/C₆⁷—H (w), C₄ₐ⁵—H/C₂⁵—H (s), C₅⁵—H/C₆⁵—OMe (s), C₄⁷—H/C₂⁷—H (s), C₄⁷—H/C_{1b}⁷—H (s), C₄⁷—H/C₁ₐ⁷—H (w), C₄⁷—H/C_{5b}⁷—OMe (s), C₄⁷—H/C₆⁷—H (w), C₆⁷—H/C₂⁵—H (w), C₆⁷—H/C_{5b}⁷—OMe (s), C₆⁷—H/C₅ₐ⁷—OMe (s), C₂⁵—H/C₃⁶—H (m), C₂⁵—H/C₂⁶—H (s), C₃⁶—H/C₂⁶—H (m), C₁⁷—(MEM-CH₂)₁/C₁ₐ⁷—H (s), C₁⁷-(MEM-CH₂)₁⁷-(MEM-CH₂)₂ (s), C₂⁷—H/C_{1b}⁷—H (s), C₂⁷—H/C₁ₐ⁷—H (s) and no NOE crosspeaks were observed between C_{5b}⁶—H/C₃⁶—H, C_{5b}⁶/C₂⁶—H, C₂⁶—H/C₃⁶—OH, N₁⁶—H/N₁⁷—H, N₁⁶—H/C₂⁵—H, and N₁⁶—H/C₄ₐ⁵—H. Most important in this spectroscopic assessment was not only the expected confirmation of the CD and AB atropisomer stereochemistry, but also the establishment of a vancomycin-like conformation for 26 bearing a cis amide linking the residues 5 and 6 (strong diagnostic C₂⁵—H/C₂⁶—H NOE) maintaining the spatial relationships and orientations of the AB ring system (strong diagnostic C₂⁵—H/C₄ₐ⁵—H and C₂⁶—H/C₄ₐ⁵—H NOEs) and CD ring systems. (diagnostic C_{6b}⁶—H/C₄ₐ⁴—H NOE). Although this might be considered unusual on the surface, even the natural atropisomer of the isolated AB ring system of vancomycin, without the surrounding CD ring system, adopts a conformation incorporating this cis amide structure illustrating that it is the confines of the AB ring system, not that of the CD ring system, that defines this key cis amide conformational preference (Boger, D. L.; et al. J. Am. Chem. Soc. 1999, 121, 3226; Boger, D. L.; et al. J. Am. Chem. Soc. 1999, 121, 10004). The lack of discernable NOEs to the methyl carbamate protecting the amine of the modified amide established that it extends out and away from the ABCD ring system binding pocket.

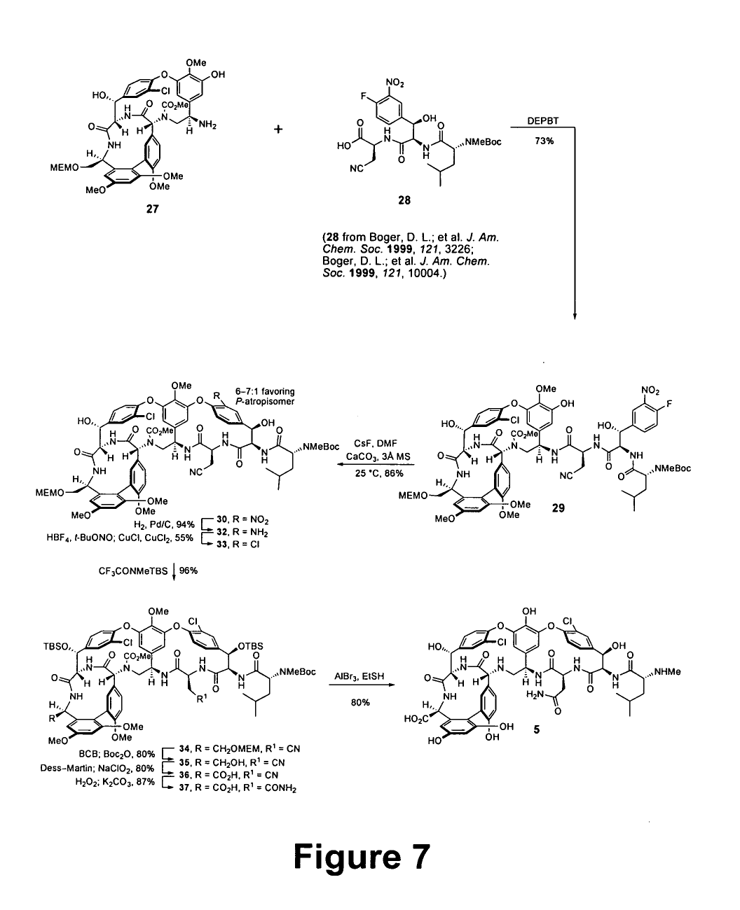

Synthesis of the Full Carbon Skeleton. Coupling of 27 and 28 (2.0 equiv of. DEPBT (Fan, C.-X.; et al. Org. Lett. 1999, 1, 91), 2.2 equiv of NaHCO₃, THF, 0-25° C., 14 h, 73%) afforded 29 with excellent diastereoselectivity (12:1) arising from little competitive racemization, FIG. 7. These conditions were utilized based on experience with the teicoplanin (Boger, D. L.; et al. J. Am. Chem. Soc. 2000, 122, 7416; Boger, D. L.; et al. J. Am. Chem. Soc. 2001, 123,1862) and ristocetin (Crowley, B. M.; et al. J. Am. Chem. Soc. 2004, 126, 4310) aglycons and are superior to those originally reported for vancomycin (Boger, D. L.; et al. J. Am. Chem. Soc. 1999, 121, 3226; Boger, D. L.; et al. J. Am. Chem. Soc. 1999, 121, 10004) (EDCl) in terms of diastereoselectivity (12:1 vs 3:1). Closure of the DE ring system with formation of the key biaryl ether was accomplished by treatment of 29 with CsF (10 equiv, 20 equiv of CaCO₃ (Both the added 3 Å MS and CaCO₃ result in cleaner conversions to product. It is not yet clear whether the soluble base under these conditions is CsF or Cs₂CO₃ with precipitation of insoluble CaF₂.), 3 Å MS, DMF, 25° C., 17 h) to afford 30 in good yield (74%) and good atropodiastereoselectivity (6-7:1). Notably, the closure of 30 was conducted under milder conditions than those originally disclosed for vancomycin (Boger, D. L.; et al. J. Am. Chem. Soc. 1999, 121, 3226; Boger, D. L.; et al. J. Am. Chem. Soc. 1999, 121, 10004; Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920; Boger, D. L.; et al. Bioorg. Med. Chem. Lett. 1995, 5, 3091; Evans, D. A.; Watson, P. S. Tetrahedron Lett. 1996, 37, 3251; Evans, D. A.; et al. Angew. Chem., Int. Ed. 1998, 37, 2700; Evans, D. A.; et al. Angew. Chem,. Int. Ed. 1998, 37, 2704) (DMF vs DMSO at 25° C. with added 3 Å MS and CaCO₃) and approaches the kinetic atropisomer diastereoselectivity observed in earlier efforts (Boger, D. L.; et al. J. Am. Chem. Soc. 1999, 121, 3226; Boger, D. L.; et al. J. Am. Chem. Soc. 1999, 121, 10004) (8:1), while surpassing that detailed in the related Evans (Evans, D. A.; et al. Angew. Chem., Int. Ed. 1998, 37, 2700; Evans, D. A.; et al. Angew. Chem,. Int. Ed. 1998, 37, 2704) efforts (5:1), and contrasts the closure detailed by Nicolaou (Nicolaou, K. C.; et al. Angew. Chem., Int. Ed. 1998, 37, 2717; Nicolaou, K. C.; et al. Angew. Chem., Int. Ed. 1998, 37, 2708; Nicolaou, K. C.; et al. Angew. Chem., Int. Ed. 1998, 37, 2714; Nicolaou, K. C.; et al. Angew. Chem., Int. Ed. 1999, 38, 240; Nicolaou, K. C.; et al. Chem. Eur. J. 1999, 5, 2584; Nicolaou, K. C.; et al. Chem. Eur. J. 1999, 5, 2602; Nicolaou, K. C.; et al. Chem. Eur. J. 1999, 5, 2622; Nicolaou, K. C.; et al. Chem. Eur. J. 1999, 5, 2648) (1:3) where the unnatural atropisomer predominated with an alternative substrate and method of ring closure. Thus, consistent with the adoption of a vancomycin-like conformation by 26, the amide modification in the ABCD ring system of 29 had little impact on the ease or diastereoselectivity of the DE ring closure. Reduction of the nitro group (Reduction of the nitro group was very sensitive to the choice of solvent in terms of recovery and observance of side products.) (H₂, 10% Pd/C, THF, 8 h, 94%) followed by diazotization of the resulting amine 32 (1.2 equiv of HBF₄, 1.2 equiv of t-BuONO, CH₃CN, 0° C., 20 min) and Sandmeyer substitution (50 equiv of CuCl, 60 equiv of CuCl₂, H₂O, 0-25° C., 1 h, 55%) gave 33, which embodies the full carbon skeleton of 5.

Completion of the Synthesis. With the full carbon skeleton in hand, attention was directed towards completion of the synthesis, FIG. 7. TBS ether protection of the secondary alcohols (65 equiv of CF₃CONMeTBS, CH₃CN, 55° C., 22 h; aq citric acid, 25° C., 13 h, 96%) followed by MEM ether deprotection of 34 (12 equiv of B-bromocatecholborane (BCB), CH₂Cl₂, 0° C., 2 h; 5.1 equiv of Boc₂O, 6.0 equiv of NaHCO₃, dioxane-H₂O 2:1, 0-25° C., 2.5 h, 80%) and two-step oxidation of the resulting primary alcohol 35 (4.0 equiv of Dess-Martin periodinane, CH₂Cl₂, 0° C., 15 min then 25° C., 1 h; 9.0 equiv of 80% aq NaClO₂, 7.0 equiv of NaH₂PO₄.H₂O, t-BuOH/2-methyl-2-butene 4:1, 25° C., 20 min, 82%) provided the carboxylic acid 36. Hydrolysis of the residue 3 nitrile with formation of the carboxamide 37 (40 equiv of 30% aq H₂O₂, 8.0 equiv of 10% aq K₂CO₃, DMSO, 25° C., 3.5 h, 87%) (Boger, D. L.; et al. J. Am. Chem. Soc. 1999, 121, 3226; Boger, D. L.; et al. J. Am. Chem. Soc. 1999, 121, 10004) set the stage for a final global deprotection (Node, M.; et al. J. Org. Chem. 1980, 45, 4275; Evans, D. A.; Ellman, J. A. J. Am. Chem. Soc. 1989, 111, 1063). In a final key reaction, 37 was treated with AlBr₃ (35 equiv, EtSH, 25° C., 5 h, 80%) to afford 5 arising from the remarkable deprotection of four aryl methyl ethers, the two TBS ethers, the N-terminus Boc group, and the critical residue 4 methyl carbamate.

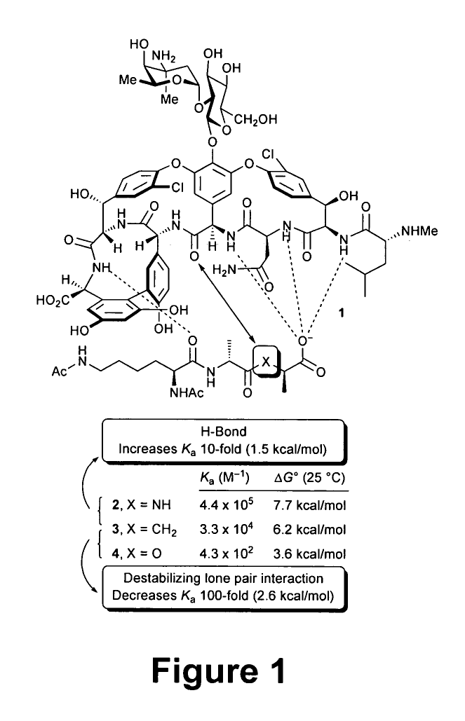

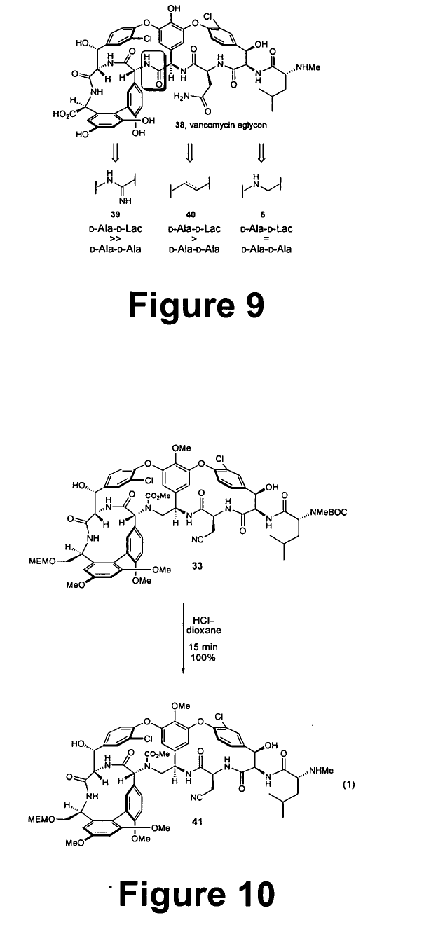

Assessment of [ψ[CH₂NH]Tpg⁴]Vancomycin Aglycon. A subtle element in the design of 5 and choice of simply removing the residue 4 carbonyl rests with the projected properties of the molecule. In principle, one might consider reengineering the capabilities of a reverse H-bond into the vancomycin structure removing the destabilizing lone pair interaction with D-Ala-D-Lac and reinstating the lost H-bond. Such opportunities include amidine derivatives (e.g. [ψ[C(═NH)NH]Tpg⁴]vancomycin aglycon, FIG. 9). Significantly, such derivatives enhance D-Ala-D-Lac binding so as to approach the level of affinity observed with vancomycin and D-Ala-D-Ala. However, such derivatives also reduce binding to D-Ala-D-Ala. Consequently, they are disclosed to gain antimicrobial activity against constitutively resistant bacteria endowed with a D-Ala-D-Lac peptidoglycan cell wall precursor (e.g. VanD), but be inactive against sensitive and inducibly resistant bacteria (VanA and VanB) that maintain or at least start with a D-Ala-D-Ala peptidoglycan cell wall precursor. The closest modified vancomycins that would be expected to reproduce the binding results observed in FIG. 1 are those that replace the amide bond linking residues 4 and 5 with a methylene (CH₂CH₂) or ethylene (CH═CH) linker. Such derivatives, by analogy with the results in FIG. 1, would be expected to enhance D-Ala-D-Lac affinity 100-fold missing only the contribution to binding derived from the H-bond.

The targeted analogue 5 incorporating an amine in the linkage of residue 4 with residue 5 not only removes the offending carbonyl and the destabilizing lone pair interaction with D-Ala-D-Lac, but it maintains a local polar environment (protonated amine) that better accommodates the binding of an electronegative group or atom (NH of D-Ala-D-Ala amide or O of D-Ala-D-Lac ester). It is disclosed herein that, while this does not bind D-Ala-D-Lac quite as well as derivatives such as 40, it is better than 40 at binding D-Ala-D-Ala.

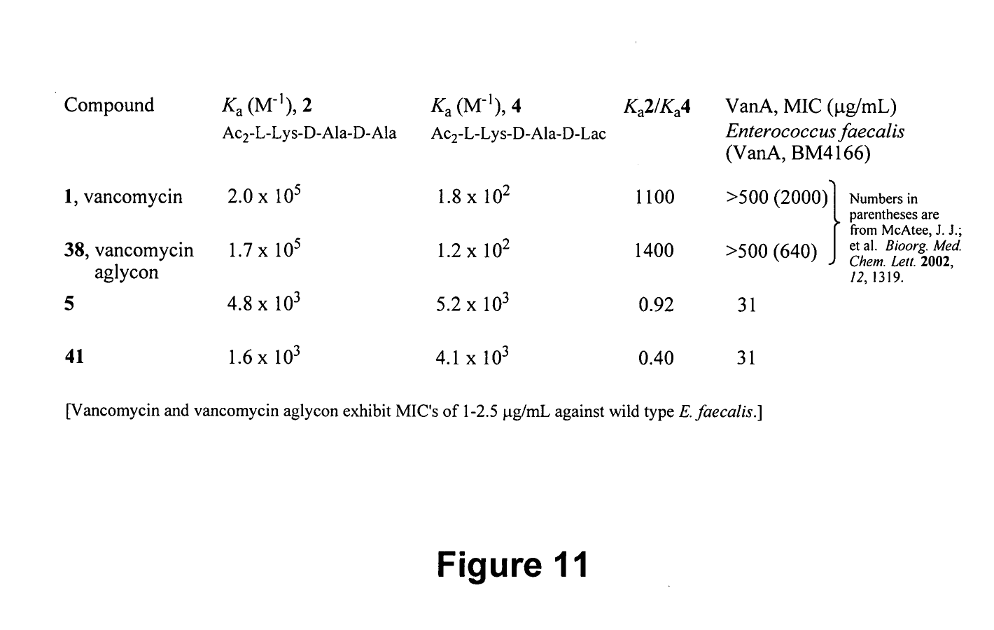

The results of the assessment of 5 alongside vancomycin (1) and its aglycon 38 are compiled in FIG. 11. An additional analogue 41, derived from N-Boc deprotection of the synthetic intermediate 33 (FIG. 10), was also examined that bears the methoxycarbonyl protecting group on the residue 4/5 linking amine. The binding affinity of 5 for Ac₂-L-Lys-D-Ala-D-Ala (2) and AC₂-L-Lys-D-Ala-D-Lac (4) was essentially equivalent (4.8 vs 5.2×10³ M⁻¹, respectively) with the D-Ala-D-Lac binding being slightly better. This represented the desired results relative to the vancomycin aglycon where the enhancement for binding D-Ala-D-Lac is 43-fold (5.2×10³ vs 1.2×10² M⁻¹) and the reduction in binding affinity for D-Ala-D-Ala is 37-fold (4.8×10³ vs 1.7×10⁵ M⁻¹). In addition, the comparison of 5 with 41 reflect the impact of the polar amine (protonated) versus its carbamate derivative where the binding affinity for D-Ala-D-Ala with 5 versus 41 increases 3-fold (4.8 vs 1.6×10³ M⁻¹) while the impact on D-Ala-D-Lac is a more marginal 1.2-fold increase in affinity (5.2 vs 4.1×10³ M⁻¹). Although there are additional structural features in the comparison of 5 and 41 that might impact the absolute affinities measured, in both instances the binding increases with the free amine 5 and it is with 5 that the dual binding is balanced.

The four compounds were compared in an antimicrobial assay against VanA Enterococcus faecalis (BM4166) that is inducibly resistant to treatment by glycopeptide antibiotics including vancomycin and teicoplanin, FIG. 11. It is the most difficult of the resistant organisms to treat (vs VanB) and characteristic of such organisms, they grow unchallenged enlisting a D-Ala-D-Ala peptidoglycan cell wall precursor, but switch to D-Ala-D-Lac upon glycopeptide treatment. As such, it represents a superb test of whether 5 and related dual D-Ala-D-Ala/D-Lac binding antibiotics might prove useful in the treatment of resistant bacteria. Compound 5 as well as 41 exhibited MICs of 31 μg/mL being roughly 40-fold more potent than vancomycin or its aglycon (MICs=2000 and 640 μg/mL) correlating well with the ca. 40-fold increase in binding affinity for D-Ala-D-Lac. Moreover, this potency is roughly 30-fold weaker than that observed with vancomycin and its aglycon against sensitive E. faecalis (MICs=1-2.5 μg/mL) correlating with the 35 to 40-fold loss in binding affinity for D-Ala-D-Ala. These results suggest that regardless of the peptidoglycan cell wall precursor utilized by the organism, it remains equally sensitive to treatment by 5 and 41.

Experimental

Compound (9): A solution of 7 (Compound 7 is available in 6 steps (37% overall) from methyl gallate using 3 recrystallizations and was scaled to 300 g, (Crowley, B. M.; et al. J. Am. Chem. Soc. 2004, 126, 4310).) (16.85 g, 35.1 mmol) in anhydrous CH₂Cl₂ (351 mL) at 0° C. under Ar was treated with Dess-Martin periodinane (29.73 g, 70.2 mmol, 2.0 equiv) and the reaction mixture allowed to slowly warm to 25° C. and stirred for 1 h. After this time, the reaction mixture was diluted with Et₂O (500 mL), quenched by addition to a cold solution of saturated aqueous NaHCO₃ (1.10 L) and saturated aqueous Na₂SO₃ (110 mL) containing Na₂S₂O₃.5H₂O (24.2 g), and stirred until two distinct layers were observed. The layers were separated and the aqueous phase extracted with Et₂O (3×700 mL). The combined organic phases were washed with cold saturated aqueous NaHCO₃ (1×700 mL) and cold saturated aqueous NaCl (1×700 mL), dried (Na₂SO₄), and the solvent was evaporated in vacuo to afford crude aldehyde 8 (16.78 g, 16.78 g theoretical, 100%) as white foam that was carried directly to the next step. Note: To prevent polymerization, the workup was carried out as quickly as possible; the product was removed from the rotary evaporator immediately upon the formation of the foam and was not dried under high vacuum, and the crude aldehyde was immediately dissolved in anhydrous CH₃OH (200 mL) upon removal from the rotary evaporator. A solution of freshly prepared 6 (Compound 6 is available in 5 steps (55% overall) from (R)-4-hydroxyphenyl-glycine using 2 recrystallizations and was scaled to 60 g, (Boger, D. L.; et al. J. Org. Chem. 1997, 62, 4721).) (11.35 g, 41.4 mmol, 1.2 equiv) and aldehyde 8 (16.78 g, 34.5 mmol) in anhydrous CH₃OH (351 mL) at 0° C. under Ar was treated with 3 Å molecular sieves (52 g, 3.0 w/w, powder) and the reaction mixture allowed to stir for 1 h. The solution was cooled to −20° C. and treated dropwise with glacial acetic acid (5.76 mL, 103.5 mmol, 3.0 equiv) to adjust the solution to pH 6 followed by portion-wise addition of NaBH₃CN (6.63 g, 103.5 mmol, 3.0 equiv, 4 equal portions, 15 min between additions). The resulting reaction mixture was stirred at −20° C. for 2 d. The reaction mixture was quenched by slow addition to cold saturated aqueous NaHCO₃ (1.0 L) and the aqueous phase extracted with EtOAc (3×800 mL). The combined organic phases were washed with saturated aqueous NaCl (800 mL), dried (Na₂SO₄), and concentrated in vacuo. Flash chromatography (SiO₂, 22.5×22.5 cm, 0-50% EtOAc-hexanes) gave 9 (19.40 g, 25.85 g theoretical, 75%) as a light yellow solid: mp 61° C.; [α]²⁵_{D}−27 (c 1.7, CHCl₃); MALDI-FTMS (DHB) m/z 757.2118 (M⁺+Na, C₃₈H₄₃BrN₂O₈ requires 757.2095).

Compound (10): A solution of 9 (15.40 g, 20.9 mmol) in anhydrous THF (420 mL) at 0° C. under Ar was treated sequentially with K₂CO₃ (28.9 g, 209 mmol, 10.0 equiv) and methyl chloroformate (16.2 mL, 209 mmol, 10.0 equiv). The reaction mixture was allowed to warm to 25° C. and stirred for 18 h. After this time, the reaction mixture was diluted with cold H₂O (300 mL) and the aqueous phase was extracted with EtOAc (3×300 mL). The combined organic phases were washed with saturated aqueous NaCl (300 mL), dried (Na₂SO₄), and concentrated in vacuo. Flash chromatography (SiO₂, 22.5×22.5 cm, 0-5% EtOAc-CH₂Cl₂; then 15% EtOAc-CH₂Cl₂) afforded 10 (14.12 g, 16.62 g theoretical, 85%) as a white solid: mp 73° C.; [α]²⁵_{D}−47 (c 0.5, CHCl₃); MALDI-FTMS (DHB) m/z 815.2150 (M⁺+Na, C₄₀H₄₅BrN₂O₁₀ requires 815.2150).

Compound (11): A solution of 10 (12.50 g, 15.7 mmol) in CH₃OH (525 mL) at 0° C. was treated with Raney nickel and the reaction mixture stirred under an atmosphere of H₂ at 0° C. for 5 h. The mixture was filtered through a pad of Celite (CH₃OH, 50 mL) and the solvent was evaporated in vacuo to give 11 (9.50 g, 9.69 g theoretical, 98%) as a white solid that was >98% pure by ¹H NMR analysis: mp 91° C.; [α]²⁵-25 (c 0.2, CH₃OH); ESI-TOF HRMS m/z 613.1395 (M⁺+H, C₂₆H₃₃BrN₂O₁₀ requires 613.1391).

Compound (S1): A solution of 10 (0.22 g, 0.27 mmol) in THF (14 mL) at 0° C. was treated with 0.2 N aqueous LiOH (1.65 mL, 0.33 mmol, 1.1 equiv) and allowed to stir for 2 h. The reaction mixture was quenched by the addition of 0.2 N aqueous HCl until the pH of the solution reached 3 and the aqueous phase extracted with EtOAc (3×10 mL). The combined organic phases were washed with saturated aqueous NaCl (1×20 mL), dried (Na₂SO₄), and concentrated in vacuo. Flash chromatography (SiO₂, 5×20 cm, 5-20% CH₃OH—CH₂Cl₂) afforded S1 (0.18 g, 0.18 g theoretical, 100%) as a white solid: mp 97° C.; [α]²⁵_{D}−126 (c 0.6, CHCl₃); MALDI-FTMS (DHB) m/z 778.2100 (M⁺+Na, C₃₉H₄₃BrN₂O₁₀ requires 778.6801).

Compound (12): A solution of 11 (9.50 g, 15.5 mmol) in THF (310 mL) at 0° C. was treated with 0.2 N aqueous LiOH (93.0 mL, 46.5 mmol, 3.0 equiv) and allowed to stir for 2 h. The mixture was quenched by the addition of 0.2 N aqueous HCl until the pH of the solution reached 3 and the aqueous phase extracted with EtOAc (3×200 mL). The combined organic phases were washed with saturated aqueous NaCl (1×200 mL), dried (Na₂SO₄), and concentrated in vacuo to give 12 (9.26 g, 9.26 g theoretical, 100%) as a white solid: mp 110° C.; [α]²⁵_{D}−57 (c 0.3, CHCl₃); MALDI-FTMS (DHB) m/z 621.1068 (M⁺+Na, C₂₅H₃₁BrN₂O₁₀ requires 621.1054).

Compound (14): A solution of 12 (2.37 g, 3.95 mmol) in anhydrous DMF (32 mL) at 0° C. under Ar was treated sequentially NaHCO₃ (1.00 g, 11.9 mmol, 3.0 equiv), DEPBT (3.55 g, 11.9 mmol, 3.0 equiv), and a solution of 13 [Compound 13 is available in 3 steps (45% overall) from 4-fluoro-3-nitrobenzaldehyde and was scaled to 30 g, (Crowley, B. M.; et al. J. Am. Chem. Soc. 2004, 126, 4310).] (1.19 g, 4.35 mmol, 1.1 equiv) in anhydrous DMF (8.0 mL). The reaction mixture was allowed to slowly warm to 25° C. and stirred for 8 h. The reaction mixture was quenched by addition to saturated aqueous NaHCO₃ (60 mL) and the aqueous phase extracted with EtOAc (3×60 mL). The combined organic phases were washed with saturated aqueous NaHCO₃ (3×60 mL), H₂O (1×60 mL), and saturated aqueous NaCl (1×60 mL), dried (Na₂SO₄), and concentrated in vacuo. Flash chromatography (SiO₂, 6×20 cm, 0-70% EtOAc-CH₂Cl₂) afforded 14 (2.65 g, 3.79 g theoretical, 70%) as a pale yellow solid: mp 121° C.; [α]²⁵_{D}−9 (c 0.8, CHCl₃); MALDI-FTMS (DHB) m/z 975.2476 (M⁺+Na, C₄₁H₅₄BrFN₄O₁₄Si requires 975.2465).

Compound (15):

Method A. A solution of 14 (2.65 g, 2.78 mmol) in anhydrous THF (230 mL, additionally dried over 3 Å MS for 18 h, then Na for 12 h) under Ar was treated with K₂CO₃ (9.60 g, 69.5 mmol, 25 equiv, dried in vacuo at 130° C. for 18 h), CaCO₃ (6.95 g, 69.5 mmol, 25 equiv, dried in vacuo at 130° C. for 18 h), and 3 Å molecular sieves (7.95 g, 3.0 w/w, powder, dried in vacuo at 130° C. for 18 h). The reaction mixture was warmed at 75° C. (bath temp.) and stirred for 12 h. After this time, the reaction mixture was cooled to 25° C. and filtered through Celite (eluted with THF) and concentrated in vacuo. The remaining solid was dissolved in EtOAc (200 mL), washed with saturated aqueous NH₄Cl (1×50 mL) and saturated aqueous NaCl, dried (Na₂SO₄), and concentrated in vacuo. Flash chromatography (SiO₂, 6×20 cm, 0-70% EtOAc-CH₂Cl₂) afforded 15 (1.40 g, 2.59 g theoretical, 54%) and 16 (0.56 g, 2.59 g theoretical, 22%) as light yellow solids (76% total conversion, 2.5:1 15:16): For 15: mp 205° C.; [α]²⁵_{D}−32 (c 1.1, CH₃OH); MALDI-FTMS (DHB) m/z 955.2379 (M⁺+Na, C₄₁H₅₃BrN₄O₁₄Si requires 955.2403).

For 16: mp 207° C.; [α]²⁵_{D}−35 (c 0.08, CH₃OH); MALDI-FTMS (DHB) m/z 955.2401 (M⁺+Na, C₄₁H₅₃BrN₄O₁₄Si requires 955.2403).

Method B. A solution of 14 (9.0 mg, 9.4 μmol) in anhydrous THF (1.57 mL, additionally dried over 3 Å MS for 18 h, then Na for 12 h) under Ar was cooled to −78° C. and treated with a freshly prepared solution of potassium tert-butoxide (1.0 mg, 9.4 μmol) in anhydrous THF (9.4 μL), and the mixture was warmed to −20° C. and stirred for 18 h. The reaction mixture was quenched by addition to cold saturated aqueous NH₄Cl (3.0 mL) and the aqueous phase was extracted with EtOAc (3×3 mL). The combined organic phases were washed with saturated aqueous NaCl (1×3 mL), dried (Na₂SO₄), and concentrated in vacuo. PTLC (SiO₂, 2.5% CH₃OH—CH₂Cl₂) afforded 15 (5.0 mg, 8.8 mg theoretical, 57%) and 16 (1.7 mg, 8.8 mg theoretical, 19%) as light yellow solids (76% total conversion, 2.9:1 15:16).

A Summary of Conditions Initially Surveyed for the Conversion of 14 to 15 may be Found in FIG. 5a and Selected Conditions Examined During the Optimization of the Reaction may be Found in FIG. 5b.

Compound (17): A solution of 15 (1.54 g, 1.65 mmol) in CH₃OH (54 mL) was treated with Raney nickel at 0° C. and the reaction mixture was stirred under an atmosphere of H₂ at 0° C. for 2 h. The reaction mixture was filtered through a pad of Celite (eluted with CH₃OH) and the solvent was removed in vacuo to afford the crude aniline (1.49 g, 1.49 g theoretical, 100%) as a light yellow foam solid that was carried on to the next step without purification. A solution of the crude aniline (1.49 g, 1.65 mmol) in anhydrous CH₃CN (26 mL, degassed) at 0° C. under Ar was treated with HBF₄ (0.1 M solution in CH₃CN, 19.4 mL, 2.15 mmol, 1.3 equiv, 0° C.) for 10 min before the dropwise addition of t-BuONO (0.1 M solution in CH₃CN, 19.4 mL, 2.15 mmol, 1.3 equiv, 0° C.). The reaction mixture was stirred at 0° C. for 10 min and then cooled to −20° C. before addition to a vigorously stirring aqueous solution (52 mL, degassed) containing CuCl (7.63 g, 82.5 mmol, 50 equiv) and CuCl₂ (12.37 g, 99 mmol, 60 equiv) at 0° C. under Ar. The resulting reaction mixture was allowed to warm to 25° C. and stirred for 1 h. The reaction mixture was quenched by slow addition to a cold saturated aqueous solution of NaHCO₃ (100 mL) and cold EtOAc (100 mL) was added before filtration through a pad of Celite. The layers were separated and the aqueous phase extracted with cold EtOAc (2×100 mL). The combined organic phases were washed with cold saturated aqueous NaHCO₃ (2×50 mL) and cold saturated aqueous NaCl (1×50 mL), dried (Na₂SO₄), and concentrated in vacuo. Flash chromatography (SiO₂, 6×20 cm, 0-70% EtOAc-CH₂Cl₂) afforded 17 (1.07 g, 1.52 g theoretical, 70%) as a light yellow solid: mp 195° C. (dec); [α]²⁵_{D}+20 (c 0.8, CHCl₃); MALDI-FTMS (DHB) m/z 944.2154 (M⁺+Na, C₄₁H₅₃BrClN₃O₁₂Si requires 944.2162).

Compound (18): A solution of 16 (5.1 mg, 5.5 mmol) in CH₃OH (1.8 mL) was treated with Raney nickel at 0° C. and the reaction mixture was stirred under an atmosphere of H₂ at 0° C. for 2 h. The mixture was filtered through a pad of Celite (eluted with CH₃OH) and the solvent was removed in vacuo to afford the crude aniline (5.0 mg, 5.0 mg theoretical, 100%) as a light yellow solid that was carried on to the next step without purification. A solution of the crude aniline (5.0 mg, 5.5 μmol) in anhydrous CH₃CN (0.9 mL, degassed) at 0° C. under Ar was treated with HBF₄ (0.1 M solution in CH₃CN, 0.65 mL, 71.7 μmol, 1.3 equiv, 0° C.) for 10 min before the dropwise addition of t-BuONO (0.1 M solution in CH₃CN, 0.65 mL, 71.7 μmol, 1.3 equiv, 0° C.). The reaction mixture was stirred at 0° C. for 10 min and then cooled to −20° C. before addition to a vigorously stirring aqueous solution (1.8 mL, degassed) containing CuCl (254 mg, 2.75 mmol, 50 equiv) and CuCl₂ (412 mg, 3.3 mmol, 60 equiv) at 0° C. under Ar and the reaction mixture was allowed to warm to 25° C. and stirred for 1 h. The reaction mixture was quenched by slow addition to a cold saturated aqueous solution of NaHCO₃ (4 mL) and cold EtOAc (4 mL) was added before filtration through a pad of Celite. The layers were separated and the aqueous phase extracted with cold EtOAc (2×4 mL). The combined organic phases were washed with cold saturated aqueous NaHCO₃ (2×2 mL) and cold saturated aqueous NaCl (1×2 mL), dried (Na₂SO₄), and concentrated in vacuo. Flash chromatography (PTLC, SiO₂, 5% CH₃OH—CH₂Cl₂) afforded 18 (3.8 mg, 5.1 mg theoretical, 75%) as a yellow solid: mp 190° C. (dec); [α]²⁵_{D}−35 (c 0.7, CHCl₃); MALDI-FTMS (DHB) m/z 944.2178 (M⁺+Na, C₄₁H₅₃BrClN₃O₁₂Si requires 944.2162).

Compound (19): From 18: A solution of 18 (2.2 mg, 2.6 mmol) in CH₃OH (1.5 mL) was treated with 10% Pd/C (0.4 mg, 0.2 w/w) and placed under an atmosphere of H₂ in a Parr shaker. The flask was pressurized with H₂ to 40 psi and shook for 16 h. The reaction mixture was filtered through Celite, the pad was washed with CH₃OH (5 mL), and the solvent evaporated in vacuo to afford 19 (1.8 mg, 2.1 mg theoretical, 85%) as a white solid: mp 180° C.; [α]²⁵_{D}+78 (c 0.45, CHCl₃); MALDI-FTMS (DHB) m/z 832.3417 (M⁺+Na, C₄₁H₅₅N₃O₁₂Si requires 832.3447).

Compound (19): From 17: A solution of 17 (1.2 mg, 1.3 mmol) in CH₃OH (1.5 mL) was treated with 10% Pd/C (0.3 mg, 0.2 w/w) and placed under an atmosphere of H₂ in a Parr shaker. The flask was pressurized with H₂to 40 psi and shook for 16 h. The reaction mixture was filtered through Celite, the pad was washed with CH₃OH, and the solvent evaporated in vacuo to afford 19 (0.85 mg, 1.1 mg theoretical, 80%) as a white solid identical in all respects to the material described above: mp 180° C.; [α]²⁵_{D}+75 (c 0.4, CHCl₃); MALDI-FTMS (DHB) m/z 832.3452 (M⁺+Na, C₄₁H₅₅N₃O₁₂Si requires 832.3447).

Compound (21): A suspension of 17 (820 mg, 0.89 mmol), 20 (Boger, D. L.; et al. J. Am. Chem. Soc. 1999, 121, 3226; Boger, D. L.; et al. J. Am. Chem. Soc. 1999, 121, 10004)(1.11 g, 2.40 mmol, 2.7 equiv), Pd₂(dba)₃ (244 mg, 0.27 mmol, 0.3 equiv), and tri-o-tolylphosphine (405 mg, 1.33 mmol, 1.5 equiv) in toluene (6.34 mL, degassed), CH₃OH (1.90 mL, degassed), and 1 M aqueous Na₂CO₃ (0.19 mL, 0.19 mmol, degassed) was warmed to 80° C. under Ar and stirred vigorously for 30 min. The mixture was cooled to 0° C., diluted with EtOAc (10 mL) and H₂O (10 mL) and treated with 1 N aqueous HCl (2.0 mL). The aqueous phase was extracted with EtOAc (3×10 mL), and the combined organic phases were washed with saturated aqueous NaCl (1×10 mL), dried (Na₂SO₄), and concentrated in vacuo. Flash chromatography (SiO₂, 6×22.5 cm, 0-70% EtOAc-CH₂Cl₂) provided 21 (443 mg, 1.13 g theoretical, 39%) as a white solid and 22 (577 mg, 1.13 g theoretical, 51%) as a white solid (1.13 g theoretical, 90%; typically 75-90%). For 21: mp 145° C.; [α]²⁵_{D}−13 (c 0.5, CHCl₃); MALDI-FTMS (DHB) m/z 1283.4874 (M⁺+Na, C₆₃H₈₁ClN₄O₁₉Si requires 1283.4845).

For 22: mp 139° C.; [α]²⁵_{D}+7 (c 0.6, CHCl₃); MALDI-FTMS (DHB) m/z 1283.4824 (M⁺+Na, C₆₃H₈₁ClN₄O₁₉Si requires 1283.4845).

Thermal equilibration and recycling of 22: A solution of 22 (260 mg, 0.21 mmol) in o-dichlorobenzene (15 mL, dried over 3 Å MS for 18 h) under Ar was placed into a 20 mL microwave reactor vial. The vial was loaded into a microwave reactor (Biotage Initiator) and heated to 210° C. for 5 min. Flash chromatography (SiO₂, 4.5×22.5 cm, 0-70% EtOAc-CH₂Cl₂) afforded 21 (110 mg, 260 mg theoretical, 42%) as a white solid and 22 (120 mg, 260 mg theoretical, 46%) as a white solid (230 mg recovered, 260 mg theoretical, 88% recovery).

Compound (23): A solution of 21 (585 mg, 0.46 mmol) in anhydrous THF (9.30 mL) at 0° C. under Ar was treated with Bu₄NF (1.0 M in THF, 567 μL, 0.56 mmol, 1.2 equiv) and the reaction mixture was allowed to stir at 0° C. for 10 min. The reaction mixture was quenched with the addition of saturated aqueous NH₄Cl (15 mL) and the aqueous phase was extracted with EtOAc (3×15 mL). The combined organic layers were washed with saturated aqueous NaCl (1×10 mL), dried (Na₂SO₄), and concentrated in vacuo. Flash chromatography (SiO₂, 3×22.5 cm, 0-100% EtOAc-CH₂Cl₂) afforded 23 (424 mg, 530 mg theoretical, 80%) as a white solid: mp 152° C.; [α]²⁵_{D}−53 (c 0.4, CHCl₃); MALDI-FTMS (DHB) m/z 1169.3964 (M⁺+Na, C₅₇H₆₇ClN₄O₁₉ requires 1169.3980).

Compound (24): A solution of 23 (392 mg, 0.34 mmol) in anhydrous 1% Cl₃CCO₂H—CH₃OH (17 mL) was treated with Pd/C (10%, 39 mg, 0.1 w/w) and stirred under an atmosphere of H₂ at 25° C. for 15 min. The reaction mixture was filtered through a pad of Celite, the pad washed with CH₃OH—HOAc (15 mL, 1% HOAc), and the pH of the solution adjusted to 8 with the addition of solid NaHCO₃. The solution was filtered through a pad of Celite and the solvent evaporated in vacuo. Flash chromatography (SiO₂, 3×22.5 cm, 5-15% CH₃OH—CH₂Cl₂) afforded 24 (329 mg, 346 mg theoretical, 95%) as a white foam: [α]²⁵_{D}−17 (c 0.4, CH₃OH); ESI-TOF HRMS m/z 1013.3798 (M⁺+H, C₄₉H₆₁ClN₄O₁₇ requires 1013.3793).

Compound (S2): A solution of 23 (23.7 mg, 20.7 mmol) in THF (1.03 mL) was treated with 0.2 N aqueous LiOH (114 mL, 114 mmol, 1.1 equiv) at 0° C. and the reaction mixture was allowed to stir for 1 h. The reaction mixture was diluted with H₂O (1.0 mL), quenched by addition of 0.2 N aqueous HCl until the pH of the solution reached 3, and the aqueous phase extracted with EtOAc (3×1.0 mL). The combined organic layers were washed with saturated aqueous NaCl (1×1.0 mL), dried (Na₂SO₄), and concentrated in vacuo. Flash chromatography (SiO₂, 0.5×18 cm, 5-15% CH₃OH—CH₂Cl₂) afforded S2 (23.4 mg, 23.4 mg theoretical, 100%) as a white foam: [α]²⁵_{D}−5.0 (c 0.4, CH₃OH); MALDI-FTMS (DHB) m/z 1155.3862 (M⁺+Na, C₅₆H₆₅ClN₄O₁₉ requires 1155.3824).

25. A solution of 24 (312 mg, 0.31 mmol) in THF—H₂O (15.4 mL, 10:1) was treated with 0.5 N aqueous LiOH (616 μL, 0.31 mmol, 1.0 equiv) at 0° C. and the reaction mixture was allowed to stir for 1 h. The reaction mixture was quenched by addition of 0.2 N aqueous HCl until the pH of the solution reached 3 and then was concentrated in vacuo. Flash chromatography (SiO₂, 2×18 cm, 5-20% CH₃OH—CH₂Cl₂) afforded 25 (292 mg, 307 mg theoretical, 95%) as a white foam: [α]²⁵_{D}+70 (c 0.4, CH₃OH); MALDI-FTMS (DHB) m/z 999.3618 (M⁺+H, C₄₈H₅₉ClN₄O₁₇ requires 999.3636).

26. A cold solution of 25 (273 mg, 0.27 mmol) in anhydrous CH₂Cl₂-DMF (72 mL, 5:1) was added dropwise over the course of 1.5 h to a stirring solution of PyBop (470 mg, 0.81 mmol, 3.0 equiv) and NaHCO₃ (126 mg, 1.62 mmol, 6.0 equiv) in CH₂Cl₂-DMF (200 mL, 5:1) at 0° C. under Ar. The reaction mixture was allowed to warm to 25° C. and stirred for 12 h. The reaction mixture was quenched by addition to saturated aqueous NH₄Cl (250 mL) and the aqueous phase was extracted with EtOAc (3×250 mL). The combined organic phases were washed with saturated aqueous NH₄Cl (5×100 mL) and saturated aqueous NaCl (1×80 mL), dried (Na₂SO₄), and concentrated in vacuo. Flash chromatography (SiO₂, 3.5×18 cm, 10% CH₃CN—CH₂Cl₂ then 0-10% CH₃OH—CH₂Cl₂) afforded 26 (188 mg, 268 mg theoretical, 70%) as a white solid: mp 188° C.; [α]²⁵_{D}−10 (c 0.8, CHCl₃); ESI-TOF HRMS m/z 981.3520 (M⁺+H, C₄₈H₅₇ClN₄O₁₆ requires 981.3531).

29. A solution of 26 (110 mg, 0.11 mmol) in CHCl₃ (5.6 mL) was treated with HCO₂H (5.6 mL) and stirred at 25° C. under Ar for 10 h. The reaction was quenched by the addition of saturated aqueous NaHCO₃ until the pH of the solution reached 7.5. The layers were separated and the aqueous phase was extracted with CHCl₃ (3×10 mL). The combined organic phases were washed with saturated aqueous NaCl (1×10 mL), dried (Na₂SO₄), and concentrated in vacuo to give the crude free amine 27 (82.8 mg, 98.8 mg theoretical, 84%) that was carried on without further purification: MALDI-FTMS (DHB) m/z 903.2823 (M⁺+Na, C₄₃H₄₉ClN₄O₁₄ requires 903.2826). A solution of 27 (82.8 mg, 0.09 mmol) and 28 (Boger, D. L.; et al. J. Am. Chem. Soc. 1999, 121, 3226; Boger, D. L.; et al. J. Am. Chem. Soc. 1999, 121, 10004) (69.3 mg, 0.12 mmol, 1.3 equiv) in anhydrous THF (1.88 mL) at 0° C. was treated sequentially with NaHCO₃ (39.5 mg, 0.47 mmol, 5.0 equiv) and DEPBT (84.3 mg, 0.28 mmol, 3.0 equiv), and the reaction mixture was allowed to warm to 25° C. and stirred for 14 h. The reaction was quenched by addition of saturated aqueous NH₄Cl (10 mL) and the aqueous phase was extracted with EtOAc (3×10 mL). The combined organic phases were washed with saturated aqueous NH₄Cl (5×10 mL) and saturated aqueous NaCl (1×10 mL), dried (Na₂SO₄), and concentrated in vacuo. Flash chromatography (SiO₂, 1×18 cm, 0-10% CH₃OH—CH₂Cl₂) afforded 29 (98.1 mg, 134.4 mg theoretical, 73%) as a yellowish white solid: [α]²⁵_{D}+19 (c 0.1, CHCl₃); ESI-TOF HRMS m/z 1430.5230 (M⁺+H, C₆₈H₈₁ClFN₉O₂₂ requires 1430.5241).

30. A solution of 29 (16.1 mg, 11.3 mmol) in anhydrous DMF (1.88 mL, dried over 3 Å MS for 18 h) was treated sequentially with 3 Å molecular sieves (48 mg, 3 w/w equiv), CaCO₃ (22.5 mg, 0.23 mmol, 20 equiv), and CsF (17.1 mg, 0.11 mmol, 10 equiv) at 25° C. under Ar and allowed to stir for 17 h. The reaction mixture was quenched by the addition of saturated aqueous NH₄Cl (5 mL) and the aqueous phase was extracted with EtOAc (3×5 mL). The combined organic phases were washed with saturated aqueous NH₄Cl (5×5 mL) and saturated aqueous NaCl (1×5 mL), dried (Na₂SO₄), and concentrated in vacuo. PTLC (SiO₂, 7.5% CH₃O—CH₂Cl₂) afforded 30 (11.7 mg, 15.9 mg theoretical, 74%) as a white solid and its atropisomer 31 (2.0 mg, 15.9 mg theoretical, 12%) as a white film. For 30: mp 244° C. (dec); [α]²⁵_{D}+57 (c 0.1, CHCl₃); ESI-TOF HRMS m/z 1410.5205 (M⁺+H, C₆₈H₈₀ClN₉O₂₂ requires 1410.5179).

For 31: [α]²⁵_{D}+15 (c 0.5, CHCl₃); ESI-TOF HRMS m/z 1410.5181 (M⁺+H, C₆₈H₈₀ClN₉O₂₂ requires 1410.5179).

33. A solution of 30 (6.8 mg, 4.8 μmol) in anhydrous THF (482 μL) was treated with 10% Pd/C (1.4 mg, 0.2 w/w equiv) and stirred under an atmosphere of H₂ in the dark for 8 h. The reaction mixture was filtered through a pad of Celite, the pad was washed with THF (15 mL), and the solvent was evaporated in vacuo at 4° C. to give crude aniline 32 (6.2 mg, 6.6 mg theoretical, 94%) that was carried on without purification. A solution of 32(6.2 mg, 4.5 μmol) in CH₃CN (448 μL, degassed) at 0° C. was treated dropwise with HBF₄ (0.1 M solution in CH₃CN, 57 μL, 5.7 μmol, 1.3 equiv, 0° C.) before the dropwise addition of t-BuONO (0.1 M solution in CH₃CN, 57 μL, 5.7 μmol, 1.3 equiv, 0° C.). The reaction mixture was stirred at 0° C. for 10 min and then cooled to −20° C. before addition to a vigorously stirring aqueous solution (900 μL, degassed) containing CuCl (22.2 mg, 225 μmol, 50 equiv) and CuCl₂ (21.7 mg, 270 μmol, 60 equiv) at 0° C. under Ar. The reaction mixture was warmed to 25° C. and stirred for 1 h. The reaction was quenched by addition to cold saturated aqueous NaHCO₃ (2 mL) and the aqueous phase extracted with EtOAc (3×2 mL). The combined organic phases were washed with cold saturated aqueous NaHCO₃ (1×2 mL) and cold saturated aqueous NaCl (1×2 mL), dried (Na₂SO₄), and the solvent removed in vacuo. PTLC (SiO₂, 7.5% CH₃OH—CH₂Cl₂) afforded 33 (1.9 mg, 3.6 mg theoretical, 54%) as a white solid: [α]²⁵_{D}+38 (c 0.1, CHCl₃); ESI-TOF HRMS m/z 1399.4924 (M⁺+H, C₆₈H₈₀Cl₂N₈O₂₀ requires 1399.4938).

34. A solution of 33 (3.8 mg, 2.7 μmol) in anhydrous CH₃CN (270 μL) under Ar was treated with CF₃CONMeTBS (42 μL, 0.18 mmol, 65 equiv), warmed to 55° C., and allowed to stir for 2 d. The reaction mixture was cooled to 25° C., quenched by addition of EtOAc-15% aqueous citric acid (4 mL, 4:1), and stirred for 15 h. The solution was diluted with H₂O (2 mL) and the aqueous phase was extracted with EtOAc (3×2 mL). The combined organic phases were washed with saturated aqueous NaHCO₃ (1×2 mL) and saturated aqueous NaCl (1×2 mL), dried (Na₂SO₄), and concentrated in vacuo. PTLC (SiO₂, 7.5% CH₃OH—CH₂Cl₂) afforded 34 (4.2 mg, 4.4 mg theoretical, 96%) as a light yellow solid: [α]²⁵_{D}+28 (c 0.1, CHCl₃); ESI-TOF HRMS m/z 1627.6685 (M⁺+H, C₈₀H₁₀₈Cl₂N₈O₂₀Si₂ requires 1627.6668).

35. A solution of 34 (2.4 mg, 1.5 μmol) in anhydrous CH₂Cl₂ (74 μL) at 0° C. under Ar in the dark was treated with B-bromocatecholborane (0.2 M solution in CH₂Cl₂, 88 μL, 18 μmol, 12 equiv) and the reaction mixture was allowed to stir for 2 h at 0° C. The reaction mixture was diluted with CH₂Cl₂ (1 mL), quenched by addition of saturated aqueous NaHCO₃ (1 mL), and the aqueous phase was extracted with EtOAc (3×1 mL). The combined organic phases were washed with saturated aqueous NaCl (1×1 mL), dried (Na₂SO₄), and the solvent evaporated in vacuo. The crude amino alcohol was dissolved in dioxane-H₂O (150 μL), cooled to 0° C., and treated sequentially with NaHCO₃ (0.7 mg, 9.0 μmol, 6.0 equiv) and Boc₂O (1.6 mg, 7.5 μmol, 5 equiv). The resulting reaction mixture was allowed to warm to 25° C. and stirred for 2.5 h. After this time, the reaction mixture was diluted with H₂O (1 mL) and the aqueous phase extracted with EtOAc (3×1 mL). The combined organic phases were washed with saturated aqueous NaCl (1×1 mL), dried (Na₂SO₄), and the solvent evaporated in vacuo. PTLC (SiO₂, 10% CH₃OH—CH₂Cl₂) afforded 35 (1.8 mg, 2.3 mg theoretical, 80%) as a white solid: [α]²⁵_{D}+18 (c 0.3, CH₃OH); ESI-TOF HRMS m/z 1561.5984 (M⁺+Na, C₇₆H₁₀₀Cl₂N₈O₁₈Si₂ requires 1561.5963).

36. A solution of 35 (1.4 mg, 0.9 μmol) in anhydrous CH₂Cl₂ (50 μL) at 0° C. under Ar was treated with Dess-Martin periodinane (1.5 mg, 3.6 μmol, 4.0 equiv) and the reaction mixture was allowed to stir for 1.5 h. The reaction mixture was diluted with Et₂O (1 mL), quenched by addition of saturated aqueous NaHCO₃ (1 mL), and the aqueous phase extracted with Et₂O (3×1 mL). The combined organic phases were washed with saturated aqueous NaCl (1×1 mL), dried (Na₂SO₄), and concentrated under a stream of N₂. The crude aldehyde was dissolved in t-BuOH-2-methyl-2-butene (50 μL, 4:1) and treated with a solution containing 80% NaClO₂ (1 mg, 8.1 μmol, 9.0 equiv) and NaH₂PO₄.H₂O (0.9 mg, 6.3 μmol, 7.0 equiv) in H₂O (8.0 μL) and the reaction mixture was allowed to stir for 30 min at 25° C. The reaction mixture was diluted with H₂O (1 mL) and the aqueous phase extracted with EtOAc (3×1 mL). The combined organic phases were washed with saturated aqueous NaCl (1×1 mL), dried (Na₂SO₄), and concentrated under a stream of N₂. PTLC (10% CH₃OH—CH₂Cl₂) afforded 36 (1.1 mg, 1.4 mg theoretical, 80%) as a white solid: [α]²⁵_{D}+21 (c 0.1, CH₃OH); ESI-TOF HRMS m/z 1553.5989 (M⁺+H, C₇₆H₉₈Cl₂N₈O₁₉Si₂ requires 1553.5936).

37. A solution of 36 (1.0 mg, 0.64 μmol) in DMSO (160 μL) was treated sequentially with H₂O₂ (50% aqueous solution, 2.0 μL, 27.2 μmol, 43 equiv) and K₂CO₃ (10% aqueous solution, 7.2 μL, 5.8 μmol, 9.0 equiv) and allowed to stir for 3.5 h. The reaction mixture was diluted with EtOAc (1 mL), quenched by the addition of 0.1 N aqueous HCl (580 μL), and the aqueous phase extracted with EtOAc (3×1 mL). The combined organic phases were washed with saturated aqueous NaCl (1×1 mL), dried (Na₂SO₄), and concentrated under a stream of N₂. PTLC (SiO₂, 10% CH₃OH—CH₂Cl₂) afforded 37 (0.9 mg, 1.0 mg theoretical, 87%) as a white film: [α]²⁵_{D}+26 (c 0.3, CH₃OH); ESI-TOF HRMS m/z 1571.6046 (M⁺+H, C₇₆H₁₀₀Cl₂N₈O₂₀Si₂ requires 1571.6042).

[ψ[CH₂NH]Tpg⁴]vancomycin aglycon (5). A vial containing 37 (0.7 mg, 0.45 μmol) was treated with a solution of AlBr₃ (4.5 mg, 16.9 μmol, 38 equiv) in EtSH (23 μL) and the reaction mixture was allowed to stir for 5 h. The reaction mixture was cooled to 0° C., diluted with CHCl₃ (100 μL), quenched by the addition of CH₃OH (10 μL), and concentrated under a stream of N₂. Reverse phase chromatography (C₁₈, 100% H₂O then 50% CH₃CN—H₂O) afforded 5 (0.4 mg, 0.5 mg theoretical, 80%) as a white film: ¹H NMR (CD₃OD, 3 mm, 600 MHz) δ7.70 (br s, 1H), 7.66 (m, 1H), 7.62 (m 1H), 7.42 (m, 1H), 7.31 (m, 1H), 7.25 (br s, 2H), 7.11 (m, 1H), 6.92 (m, 1H), 6.84 (m, 1H), 6.42 (br s, 1H), 5.50 (s, 1H), 5.46 (s, 1H), 5.37 (m, 1H), 5.33 (br s, 2H), 5.26 (m, 1H), 4.91 (m, obscured by HOD, 1H), 4.52 (m, 1H), 4.21 (m, 1H), 4.11 (m, 1H), 4.07 (br s, 1H), 3.74 (m, 1H), 3.68 (s, 4H), 3.57 (br s, 5H), 2.81 (m, 2H), 2.77 (s, 3H), 2.02 (m, 1H), 2.18 (m, 1H), 1.66 (m, 1H), 1.59 (s, 2H), 0.94 (m, 3H), 0.88 (m, 3H); MALDI-TOF m/z 1129.2 (M⁺+H, C₅₃H₅₄Cl₂N₈O₁₆ requires 1129.3).

Binding Constant Determination. The binding constants for compounds 5 and 41 for association with the model ligands N,N′-Ac₂-Lys-D-Ala-D-Ala and N,N′-Ac₂-Lys-D-Ala-D-Lac were determined according to literature (Nieto, M.; Perkins, H. R. Biochem. J. 1971, 124, 845; Nieto, M.; Perkins, H. R. Biochem. J. 1971, 124, 773; Nieto, M.; Perkins, H. R. Biochem. J. 1971, 124, 789) protocols. UV difference experiments were carried out on a CARY 3E UV-Vis spectrometer. UV scans were run with a baseline correction that consisted of 0.02 M sodium citrate buffer and covered a range from 200 to 345 nm. A solution of 5 or 41 (1.1×10⁻⁴ M in 0.02 M sodium citrate buffer) was placed into a quartz UV cuvette (1.0 cm path length) and the UV spectrum recorded versus a reference cell containing 0.02 M sodium citrate buffer. UV spectra were recorded after each addition of a solution of N,N′-Ac₂-Lys-D-Ala-D-Ala or N,N′-Ac₂-Lys-D-Ala-D-Lac in 0.02 M sodium citrate buffer to each cell from 0.1 to 140.0 equivalents. The absorbance value at the λₘₐₓ was recorded and the running change in absorbance, ΔA_{x equiv} (Aᵢₙᵢₜᵢₐₗ−A_{x equiv}, ) measured. The number of ligand equivalents was plotted versus ΔA to afford the ligand binding titration curve. The break point of this curve is the saturation point of the system and its xy coordinates determined by establishing the intersection of the linear fits of the pre and postsaturation curves. ΔAₛₐₜᵤᵣₐₜᵢₒₙ was calculated and employed to determine the concentration of free ligand in solution at each titration. ΔA was plotted versus ΔA/free ligand concentration to give a Scatchard plot from which the binding constants were determined.

### DETAILED DESCRIPTION OF DRAWINGS

FIG. 1 illustrates the factors that determine the binding affinity of Vancomycin and its analogs to the model tripeptide and the rationale for the omission of the carbonyl oxygen of amino acid 4. Thus, the binding affinity of vancomycin for 3, which incorporates a methylene (CH₂) in place of the linking amide NH of Ac₂-L-Lys-D-Ala-D-Ala, was compared with that of Ac₂-L-Lys-D-Ala-D-Ala (2) and Ac₂-L-Lys-D-Ala-D-Lac (4). The vancomycin affinity for 3 was approximately 10-fold less than that of 2, but 100-fold greater than that of 4. This indicated that the reduced binding affinity of 4 (4.1 kcal/mol) may be attributed to both the loss of a key H-bond and a destabilizing lone pair/lone pair interaction introduced with the ester oxygen of 4 (2.6 kcal/mol) with the latter, not the H-bond, being responsible for the greater share (100-fold) of the 1000-fold binding reduction. These observations can be employed for the reengineering of vancomycin to bind D-Ala-D-Lac. It is disclosed herein that redesign of vancomycin focuses principally on removing the destabilizing lone pair interaction rather than reintroduction of a H-bond and that this may be sufficient to compensate for two of the three orders of magnitude in binding affinity lost with D-Ala-D-Lac. Thus, synthesis of a vancomycin analogue with removal of the residue 4 carbonyl and its destabilizing lone pair interaction restores much of the binding affinity of the antibiotic for the modified ligand. At present, such a deep-seated change in the molecule can only be achieved by total synthesis, since previous efforts to selectively modify the residue 4 carbonyl by selective reaction of the amide linking residues 4 and 5 within vancomycin aglycon derivatives have not yet been successful.

FIG. 2 illustrates the retrosynthetic steps used to map out the synthesis of this vancomycin analog. The desired analogue 5 was anticipated to be prepared by a route analogous to that developed for vancomycin (Boger, D. L.; et al. J. Am. Chem. Soc. 1999, 121, 3226; Boger, D. L.; et al. J. Am. Chem. Soc. 1999, 121, 10004), with notable modifications. Thus, two aromatic nucleophilic substitution reactions with formation of the biaryl ethers were then enlisted for CD and DE macrocyclization, a key macrolactamization reaction were employed for cyclization of the AB ring system, and the defined order of CD, AB, and DE ring closures permitted sequential control of the atropisomer stereochemistry of each of the newly formed ring systems or their immediate precursors. Thus, in addition to any kinetic diastereoselection that may be achieved in the ring closures, this order permitted the recycling of any undesired atropisomer for each newly introduced ring system by thermal equilibration providing a predictable control of the stereochemistry and dependably funneling all synthetic material into one of eight possible atropdiastereomers. Key to recognition of this preferential order of ring closures was the establishment of the thermodynamic parameters of atropisomerism for the individual vancomycin ring systems: DE ring system (Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920) (Eₐ=18.7 kcal/mol)<AB biaryl precursor (Boger, D. L.; et al. J. Am. Chem. Soc. 1999, 121, 3226; Boger, D. L.; et al. J. Am. Chem. Soc. 1999, 121, 10004) (Eₐ=25.1 kcal/mol)<CD ring system (Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920) (Eₐ=30.4 kcal/mol).

FIG. 3 is a scheme showing the synthesis of the BCD “tripeptide.” The B and D subunits 6 and 7 were prepared following previously optimized procedures (see main text for references). Oxidation of alcohol 7 (Compound 7 is available in 6 steps (37% overall) from methyl gallate using 3 recrystallizations and was scaled to 300 g, (Crowley, B. M.; et al. J. Am. Chem. Soc. 2004, 126, 4310)) (2.0 equiv of Dess-Martin periodinane, CH₂Cl₂, 0-25° C., 1 h, 100%) was followed by immediate reductive amination coupling of the sensitive aldehyde 8 with 6 (Compound 6 is available in 5 steps (55% overall) from (R)-4-hydroxyphenyl-glycine using 2 recrystallizations and was scaled to 60 g, (Boger, D. L.; et al. J. Org. Chem. 1997, 62, 4721)) (1.1 equiv, CH₃OH, 3 Å MS, 0° C., 45 min; 3.0 equiv of AcOH, 3.0 equiv of NaBH₃CN, −20° C., 2 d) to afford amine 9 in good yield (75%) and excellent diastereoselectivity (12:1). Shorter reaction times (14-20 h) at higher temperatures (−15 to −5° C.) led to substandard selectivities (4:1 to 9:1) and the use of less NaBH₃CN (1.5-2.0 equiv) at lower temperatures (−20° C.) led to incomplete reactions. Longer reaction times (3-8 d) led to only marginal increases in yield (82% after 8 d) and roughly equal diastereoselectivities. Amine protection of 9 as the methyl carbamate (10 equiv of MeOCOCl, 10 equiv of K₂CO₃, THF, 0-25° C., 18 h, 85%) followed by benzyl ether deprotection (Benzyl ether deprotection at higher temperatures (25° C.) may lead to competitive aryl bromide reduction although this was only observed in appreciable amounts when excess Raney Ni was employed.) (Raney Ni, CH₃OH, 0° C., 5 h, 98%) and saponification (3.0 equiv of LiOH, THF—H₂O, 0° C., 6 h, 100%) provided 12. Unexpectedly, the order of these latter two deprotections proved important. Saponification of 10 (Saponification of 11 was considerably slower than that of 10 and occasionally required additional LiOH for complete conversion to 12 with little effect on the amount of epimer generated in the reaction.) under a variety of conditions (LiOH, THF—H₂O or t-BuOH—H₂O, −10 to 0° C.; LiOOH, THF—H₂O; Me₃SnOH, 1,2-dichloroethane, 70° C.) led to variable amounts of an epimer (5-20%) that was difficult to separate from the product. In contrast, benzyl ether deprotection of 10 followed by saponification of 11 reduced the amount of epimer (0-3%) presumably due to preferential deprotonation of the phenols such that subsequent C_{α} deprotonation at residue 5 was less facile (Saponification of 11 was considerably slower than that of 10 and occasionally required additional LiOH for complete conversion to 12 with little effect on the amount of epimer generated in the reaction.). Coupling of 12 with 13 (Compound 13 is available in 3 steps (45% overall) from 4-fluoro-3-nitrobenzaldehyde and was scaled to 30 g, (Crowley, B. M.; et al. J. Am. Chem. Soc. 2004, 126, 4310).) (3.0 equiv of DEPBT (Fan, C.-X.; et al. Org. Lett. 1999, 1, 91), 3.0 equiv of NaHCO₃, DMF, 0-25° C., 8 h) gave “tripeptide” 14 in good yield (70%) and excellent diastereoselectivity (14:1).

FIG. 4 is a scheme for the synthesis of the ABCD ring system starting from N-Boc amino ester diamide 14. After considerable optimization (FIGS. 5A and 5B), cyclization of 14 (20 equiv of K₂CO₃, 20 equiv of CaCO₃, 3 wt equiv of 3 Å MS, 12 mM THF, 75° C. bath temp, 12 h) afforded 15 in good yield (54%) and good atropodiastereoselectivity (2.5:1, 15 (54%) and 16 (22%)) even when conducted on a large scale (2.7 g). Reduction of the nitro group (Raney Ni, 0° C., CH₃OH, 1 h) followed by diazotization (1.3 equiv of HBF₄, 1.3 equiv of t-BuONO, CH₃CN, 0° C., 30 min) and Sandmeyer substitution (50 equiv of CuCl, 60 equiv of CuCl₂, H₂O, 0-25° C., 1 h, 70% from 15) cleanly provided 17 without loss of the atropisomer stereochemistry inherent in starting 15. The unnatural atropisomer 16 was also subjected to these conditions to cleanly give 18 (75%) (FIG. 6). Suzuki coupling of 17 with the hindered A ring boronic acid 20 (Boger, D. L.; et al. J. Am. Chem. Soc. 1999, 121, 3226; Boger, D. L.; et al. J. Am. Chem. Soc. 1999, 121, 10004) (0.3 equiv of Pd₂(dba)₃, 1.5 equiv of (o-tol)₃P, toluene-CH₃OH-1 N aq Na₂CO₃ 10:3:1, 80° C., 30 min) proceeded in excellent yield (90%) under remarkably effective conditions (Boger, D. L.; et al. J. Am. Chem. Soc. 1999, 121, 3226; Boger, D. L.; et al. J. Am. Chem. Soc. 1999, 121, 10004) given the steric constraints of the substrate 20 providing a separable 1:1.3 mixture of atropisomers (21:22) slightly favoring the unnatural configuration. Thermal equilibration of isolated 22 was carried out initially employing the reported conditions for vancomycin (o-dichlorobenzene, 120° C., 18 h, 81% recovery of material) (Boger, D. L.; et al. J. Org. Chem. 1997, 62, 4721; Boger, D. L.; et al. J. Org. Chem. 1996, 61, 3561; Boger, D. L.; et al. J. Org. Chem. 1999, 64, 70; Boger, D. L.; et al. Bioorg. Med. Chem. Lett. 1997, 7, 3199; Boger, D. L.; et al. Bioorg. Med. Chem. Lett. 1998, 8, 721; Boger, D. L.; et al. J. Am. Chem. Soc. 1998, 120, 8920) to afford a 1:1.1 separable mixture permitting the recycling of this unnatural atropisomer. Silyl ether deprotection of 21 (1.2 equiv of Bu₄NF, THF, 0° C., 10 min) followed by N-Cbz removal (H₂, 10% Pd/C, 1% Cl₃CCO₂H—CH₃OH, 15 min, 95%) and methyl ester hydrolysis (1.0 equiv of LiOH, THF—H₂O, 0° C., 1.h, 96%) gave amino acid 25. Macrolactamization with closure of the AB ring system was effected by treatment of 25 with PyBOP (3.0 equiv, 6.0 equiv of NaHCO₃, 0.001 M CH₂Cl₂-DMF 5:1, 0-25° C., 12 h) to afford the fully functionalized bicyclic ABCD ring system 26 in good yield (70%) with only trace amounts of competitive epimerization (<3%).

FIG. 5A is a table summarizing the conditions tested for the cyclization of 14 to 15. The inclusion of CaCO₃ in the reaction mixture is critical and serves to trap the liberated fluoride arising from the aromatic nucleophilic substitution as an insoluble byproduct (CaF₂) preventing TBS ether deprotection and a subsequent competitive base-catalyzed retro aldol reaction of the free alcohol. The cyclization of 14 represents a considerable improvement over the analogous ring closure reaction enlisted in this inventor's original synthesis of vancomycin (50-65%, 1:1 atropisomers vs 76-87%, 2.5-3:1 atropisomers) where both the overall conversion and atropodiastereoselectivity were lower illustrating that the closure of 14 may benefit from both the increased conformational flexibility of the cyclization substrate and the residue 4 amine small protecting group.

FIG. 5B is a table summarizing the conditions used for the cyclization of 14 to 15 after conditions in FIG. 5A were tried.

FIG. 6 is a short scheme showing the steps taken to attempt to recycle the undesired atropdiastereomers 15 and 17 by heating in solvent and how they were identified as atropisomers of 16 and 18, respectively. These two compounds were shown to be atropdiastereomers of 16 and 18, respectively, by conversion of 17 to 19. The identity of compound 19 was confirmed by conversion from 18 and 17 by dechlorination/debromination. Unlike the vancomycin CD ring system in which the atropisomers could be thermally equilibrated at 120-140° C. permitting the recycling and productive use of the unnatural atropisomer, the atropisomers 15 and 16 could not be thermally interconverted even at temperatures as high as 210-230° C. The corresponding chloro compounds 17 and 18 were not able to be interconverted either.

FIG. 7 shows the synthesis of the complete carbon skeleton of the vancomycin aglycon analog. Coupling of 27 and 28 (2.0 equiv of DEPBT (Fan, C.-X.; et al. Org. Lett. 1999, 1, 91), 2.2 equiv of NaHCO₃, THF, 0-25° C., 14 h, 73%) afforded 29 with excellent diastereoselectivity (12:1) arising from little competitive racemization. Closure of the DE ring system with formation of the key biaryl ether was accomplished by treatment of 29 with CsF (10 equiv, 20 equiv of CaCO₃ (Both the added 3 Å MS and CaCO₃ result in cleaner conversions to product. It is not yet clear whether the soluble base under these conditions is CsF or Cs₂CO₃ with precipitation of insoluble CaF₂.), 3 Å MS, DMF, 25° C., 17 h) to afford 30 in good yield (74%) and good atropodiastereoselectivity (6-7:1). Thus, consistent with the adoption of a vancomycin-like conformation by 26, the amide modification in the ABCD ring system of 29 had little impact on the ease or diastereoselectivity of the DE ring closure. Reduction of the nitro group (Reduction of the nitro group was very sensitive to the choice of solvent in terms of recovery and observance of side products.) (H₂, 10% Pd/C, THF, 8 h, 94%) followed by diazotization of the resulting amine 32 (1.2 equiv of HBF₄, 1.2 equiv of t-BuONO, CH₃CN, 0° C., 20 min) and Sandmeyer substitution (50 equiv of CuCl, 60 equiv of CuCl₂, H₂O, 0-25° C., 1 h, 55%) gave 33, which embodies the full carbon skeleton of 5.

TBS ether protection of the secondary alcohols (65 equiv of CF₃CONMeTBS, CH₃CN, 55° C., 22 h; aq citric acid, 25° C., 13 h, 96%) followed by MEM ether deprotection of 34 (12 equiv of B-bromocatecholborane (BCB), CH₂Cl₂, 0° C., 2 h; 5.1 equiv of Boc₂O, 6.0 equiv of NaHCO₃, dioxane-H₂O 2:1, 0-25° C., 2.5 h, 80%) and two-step oxidation of the resulting primary alcohol 35 (4.0 equiv of Dess-Martin periodinane, CH₂Cl₂, 0° C., 15 min then 25° C., 1 h; 9.0 equiv of 80% aq NaClO₂, 7.0 equiv of NaH₂PO₄.H₂O, t-BuOH/2-methyl-2-butene 4:1, 25° C., 20 min, 82%) provided the carboxylic acid 36. Hydrolysis of the residue 3 nitrile with formation of the carboxamide 37 (40 equiv of 30% aq H₂O₂, 8.0 equiv of 10% aq K₂CO₃, DMSO, 25° C., 3.5 h, 87%) (Boger, D. L.; et al. J. Am. Chem. Soc. 1999, 121, 3226; Boger, D. L.; et al. J. Am. Chem. Soc. 1999, 121, 10004) set the stage for a final global deprotection (Node, M.; et al. J. Org. Chem. 1980, 45, 4275; Evans, D. A.; Ellman, J. A. J. Am. Chem. Soc. 1989, 111, 1063). In a final key reaction, 37 was treated with AlBr₃ (35 equiv, EtSH, 25° C., 5 h, 80%) to afford 5 arising from the remarkable deprotection of four aryl methyl ethers, the two TBS ethers, the N-terminus Boc group, and the critical residue 4 methyl carbamate.

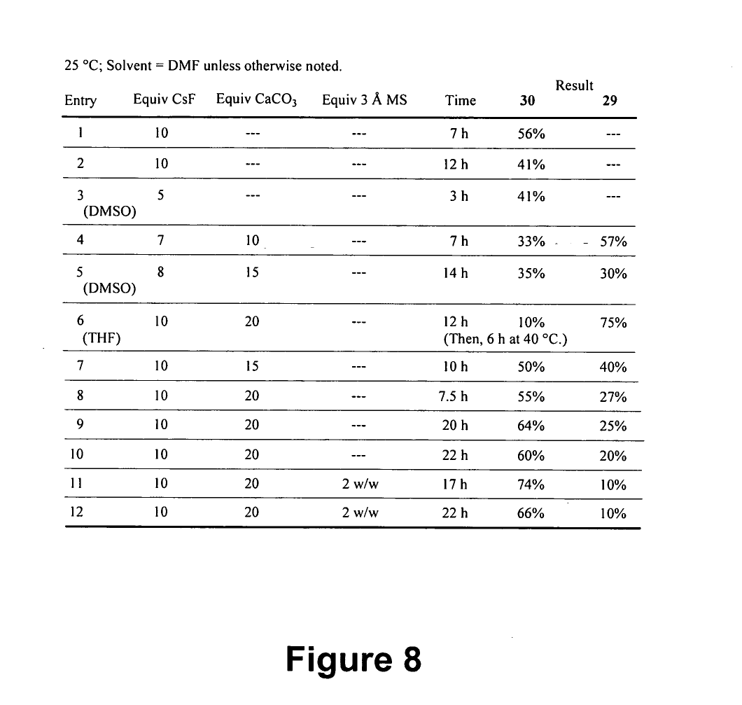

FIG. 8 is a table that shows the conditions used for the cyclization of 29 to form 30 by catalyzing with a fluoride ion in the presence of added base. Closure of the DE ring system with formation of the key biaryl ether was accomplished by treatment of 29 with CsF (10 equiv, 20 equiv of CaCO₃ (Both the added 3 Å MS and CaCO₃ result in cleaner conversions to product. It is not yet clear whether the soluble base under these conditions is CsF or Cs₂CO₃ with precipitation of insoluble CaF₂.), 3 Å MS, DMF, 25° C., 17 h) to afford 30 in good yield (74%) and good atropodiastereo-selectivity (6-7:1).

FIG. 9 is a drawing showing the different modifications in the vancomycin structure of analogs that are possible and what relative affinity they might have for either the D-Ala-D-Ala ligand or the D-Ala-D-Lac ligand. The targeted analogue 5 incorporating an amine in the linkage of residue 4 with residue 5 not only removes the offending carbonyl and the destabilizing lone pair interaction with D-Ala-D-Lac, but it maintains a local polar environment (protonated amine) that may better accommodate the binding of an electronegative group or atom (NH of D-Ala-D-Ala amide or O of D-Ala-D-Lac ester). While this might not bind D-Ala-D-Lac quite as well as derivatives such as 40, it was better than 40 at binding D-Ala-D-Ala. In the best case, 5 might bind D-Ala-D-Ala and D-Ala-D-Lac with equal affinities making it effective for the treatment of sensitive or resistant bacteria regardless of the structure of the peptidoglycan cell wall precursor.

FIG. 10 is an N-Boc deprotection of 33 to give 41 without deprotecting the methyl carbamate of residue 4 and removing the MEM group. Compound 41 was synthesized to test its binding affinity in comparison with vancomycin, 5 and 38.

FIG. 11 is a table showing the results of the assessment of 5 alongside vancomycin (1) and its aglycon 38 and structure 41. An additional analogue 41, derived from N-Boc deprotection of the synthetic intermediate 33 (FIG. 10), was also examined that bears the methoxycarbonyl protecting group on the residue 4/5 linking amine. The binding affinity of 5 for AC₂-L-Lys-D-Ala-D-Ala (2) and Ac₂-L-Lys-D-Ala-D-Lac (4) was essentially equivalent (4.8 vs 5.2×10³ M⁻¹, respectively) with the D-Ala-D-Lac binding being slightly better. Impressively, this represented the anticipated results relative to the vancomycin aglycon where the enhancement for binding D-Ala-D-Lac is 43-fold (5.2×10³ vs 1.2×10² M⁻¹) and the reduction in binding affinity for D-Ala-D-Ala is 37-fold (4.8×10³ vs 1.7×10⁵ M⁻¹).

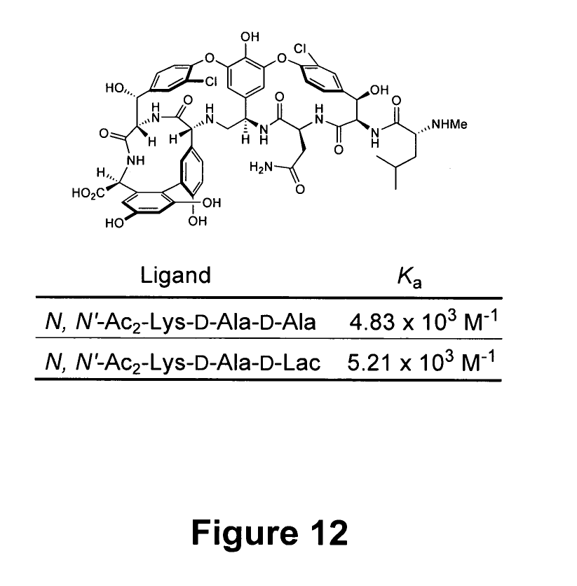

FIG. 12 shows the structure of the vancomycin analog and its binding constant with the two model ligands.

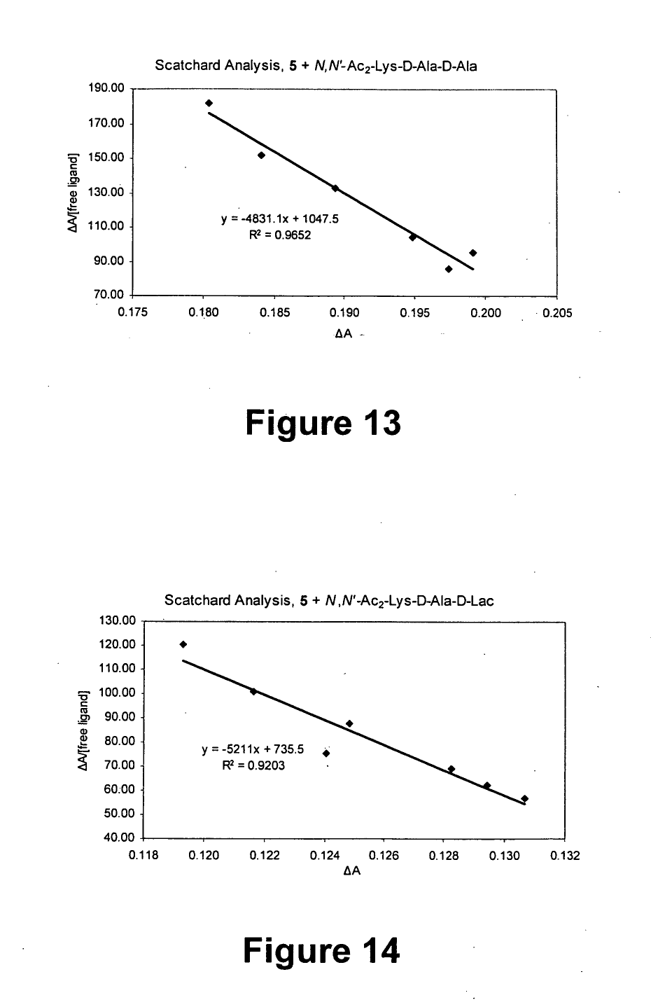

FIG. 13 is a Skatchard analysis of compound 5 with the N,N′-Ac₂-Lys-D-Ala-D-Ala ligand. The binding constants for compounds 5 and 41 for association with the model ligands N,N′-Ac₂-Lys-D-Ala-D-Ala and N,N′-Ac₂-Lys-D-Ala-D-Lac were determined according to literature (Nieto, M.; Perkins, H. R. Biochem. J. 1971, 124, 845; Nieto, M.; Perkins, H. R. Biochem. J. 1971, 124, 773; Nieto, M.; Perkins, H. R. Biochem. J. 1971, 124, 789) protocols.

FIG. 14 is a Skatchard analysis of compound 5 with the N, N′-Ac₂-Lys-D-Ala-D-Lac ligand.

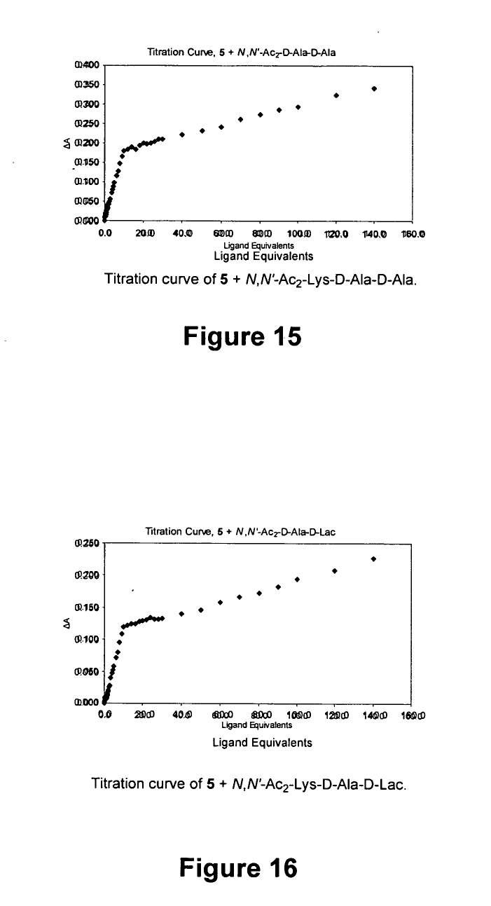

FIG. 15 is a titration curve of 5 and the N,N′-Ac₂-Lys-D-Ala-D-Ala ligand.

FIG. 16 is a titration curve of 5 and the N, N′-Ac₂-Lys-D-Ala-D-Lac ligand.

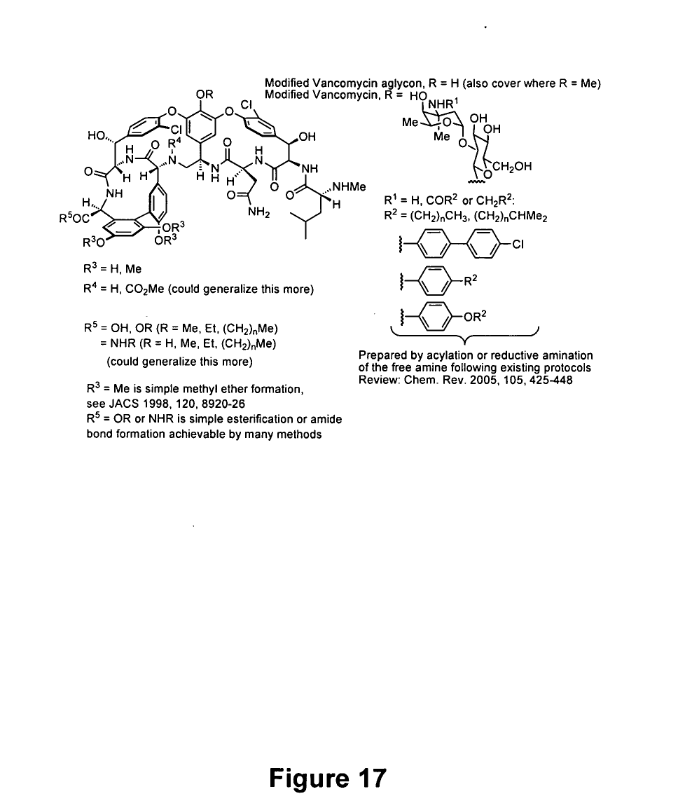

FIG. 17 illustrates important modifications to the basic vancomycin analog structure. Most of the modifications are in the peripheral portion of the molecule as the backbone of the vancomycin structure has been preserved with the exception of the carbonyl oxygen of the fourth amino acid. This carbonyl has been replaced by a methylene group eliminating an energetically unfavorable interaction with the lone pairs of the ester oxygen of the D-Lac.

What is claimed is:

1. A composition having antibacterial activity with respect to glycopeptide antibiotic resistant bacteria and dual binding activity with respect to D-Ala-D-Ala and D-Ala-D-Lac, said composition comprising a [ψ[CH₂NH]PG⁴] glycopeptide antibiotic analog or aglycon combined with a physiologically acceptable carrier.

2. A composition according to claim 1 wherein said [ψ[CH₂NH]PG⁴] glycopeptide antibiotic analog or aglycon is an analog of a glycopeptide antibiotic selected from the group consisting of vancomycins, teicoplanins, balhimycins, actinoidins, ristocetins, and orienticins or of their respective aglycons.

3. A composition according to claim 1 wherein said [ψ[CH₂NH]PG⁴] glycopeptide antibiotic analog or aglycon is a polycyclic heptapeptide having amino acids numbers 1-7,at least two macrocyclic rings, and an optional sugar unit, wherein
amino acids numbers 2, 4 and 6 of said polycyclic heptapeptide each having a side chain containing a benzene ring, amino acid number 4 being a phenyl glycine,
each of said macrocyclic rings being independently derived from a bonding together of two different benzene rings of said amino acids, either through an ether linkage or by having the benzene rings being directly bonded together through a sigma bond,
the phenyl glycine of amino acid number 4 being bonded at positions 3 and 5 to the benzene rings of the side chains of amino acids number 2 and number 6 through ether linkages or by direct sigma bonding, and
said polycyclic heptapeptide including optional further macrocyclic structures formed between the side chains of amino acids 1 and 3 and/or between the side chains of amino acids 5 and 7 through direct sigma bonds or through ether linkages.

4. A composition according to claim 3 wherein said [ψ[CH₂NH]PG⁴] glycopeptide antibiotic analog is an aglycon and lacks a sugar unit.

5. A composition according to claim 3 wherein said [ψ[CH₂NH]PG⁴] glycopeptide antibiotic analog includes at least one sugar unit.

6. A composition according to claim 3 wherein said [ψ[CH₂NH]PG⁴] glycopeptide antibiotic analog is [ψ[CH₂NH]TPG⁴] vancomycin.

7. A composition according to claim 3 wherein said [ψ[CH₂NH]PG⁴] glycopeptide antibiotic analog is [ψ[CH₂NH]TPG⁴] vancomycin aglycon.

8. A process for decreasing the viability of glycopeptide antibiotic resistant bacteria, the glycopeptide antibiotic resistant bacteria being of a type that is resistant to either D-Ala-D-Ala or D-Ala-D-Lac binding glycopeptide antibiotics but not both, the process comprising the step of contacting the bacterium with a bactericidal concentration of a [ψ[CH₂NH]PG⁴] glycopeptide antibiotic analog or aglycon, the [ψ[CH₂NH]PG⁴] glycopeptide antibiotic analog or aglycon being of a type having dual binding activity with respect to D-Ala-D-Ala and D-Ala-D-Lac and antibacterial activity with respect to said glycopeptide antibiotic resistant bacteria.

9. A process according to claim 8 wherein said [ψ[CH₂NH]PG⁴] glycopeptide antibiotic analog or aglycon is an analog of a glycopeptide antibiotic selected from the group consisting of vancomycins, teicoplanins, balhimycins, actinoidins, ristocetins, and orienticins or of their respective aglycons.

10. A process according to claim 8 wherein said [ψ[CH₂NH]PG⁴] glycopeptide antibiotic analog or aglycon is a polycyclic heptapeptide having amino acids numbers 1-7,at least two macrocyclic rings, and an optional sugar unit, wherein
amino acids numbers 2, 4 and 6 of said polycyclic heptapeptide each having a side chain containing a benzene ring, amino acid number 4 being a phenyl glycine,
each of said macrocyclic rings being independently derived from a bonding together of two different benzene rings of said amino acids, either through an ether linkage or by having the benzene rings being directly bonded together through a sigma bond,
the phenyl glycine of amino acid number 4 being bonded at positions 3 and 5 of the phenyl to the benzene rings of the side chains of amino acids number 2 and number 6 through ether linkages or by direct sigma bonding, and
said polycyclic heptapeptide including optional further macrocyclic structures formed between the side chains of amino acids 1 and 3 and/or between the side chains of amino acids 5 and 7 through direct sigma bonds or through ether linkages.

11. A compound represented by the following structure: 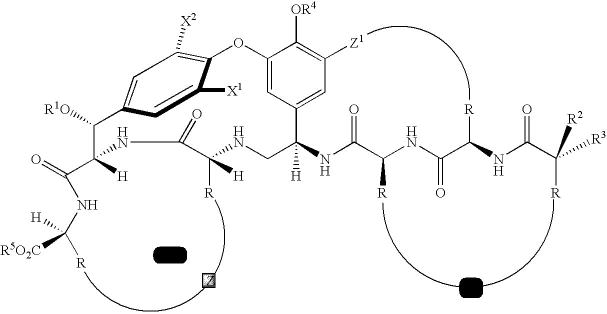
wherein:
each R is independently selected from the group consisting of amino acid side chains, phenyl rings substituted by one or more chlorines, hydroxy groups, amino groups, sulfates, and sugars;
each Z is independently either absent, a sigma bond or a bridging oxygen;
Z¹ is a sigma bond or a bridging oxygen;
X¹ is either chloro or hydrogen;
X² is either chloro or hydrogen;
R¹ is selected from the group consisting of hydrogen, sugar, amino sugar, N-alkyl (C1-C6) amino sugar, and acylated amino sugar;
R² is hydrogen or with R³ forms a carbonyl group;
R³ is selected from the group consisting of amino, methylamino, dimethylamino, and trimethylammonium, or with R² forms a carbonyl group;
R⁴ is selected from the group consisting of hydrogen, methyl, sugar, amino sugar, N-alkyl (C1-C6) amino sugar, and acylated amino sugar; and
R⁵ is selected from the group consisting of hydrogen, methyl, and C2-C6 alkyl.

12. A compound according to claim 11 represented by the following structure: 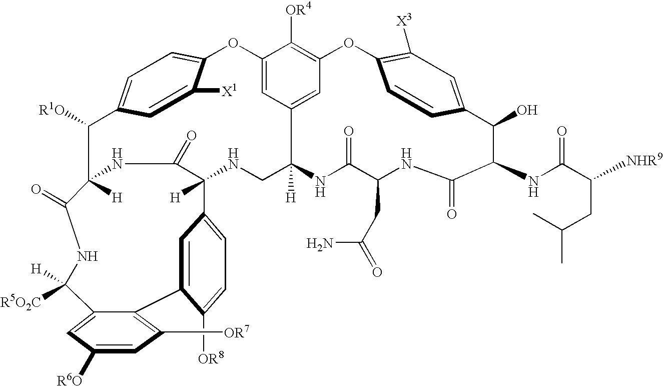
wherein:
X¹ is either chloro or hydrogen;
X³ is either chloro or hydrogen;
R¹ is selected from the group consisting of hydrogen, sugar, amino sugar, N-alkylamino sugar, and acylated amino sugar;
R⁴ is selected from the group consisting of hydrogen, methyl, sugar, amino sugar, N-alkylamino sugar, and acylated amino sugar;
R⁵ is selected from the group consisting of hydrogen, methyl, and C2-C6 alkyl;
R⁶ is selected from the group consisting of hydrogen, methyl, sugar, amino sugar, N-alkylamino sugar, and acylated amino sugar;
R⁷ is selected from the group consisting of hydrogen, methyl, sugar, amino sugar, N-alkylamino sugar, and acylated amino sugar;
R⁸ is selected from the group consisting of hydrogen, methyl, sugar, amino sugar, N-alkylamino sugar, and acylated amino sugar; and
R⁹ is hydrogen or methyl.

13. A compound according to claim 11 having the following structure: 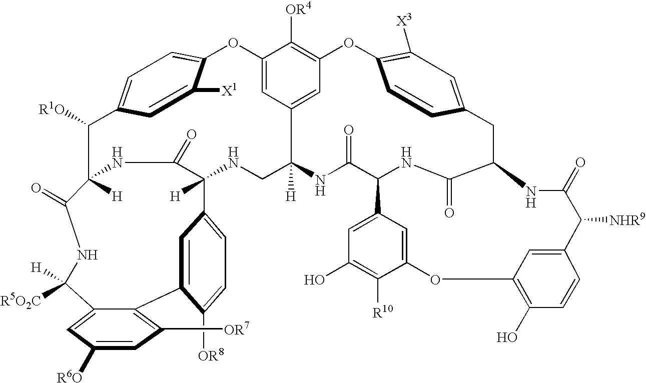
wherein
X¹ is either chloro or hydrogen;
X³ is either chloro or hydrogen;
R¹ is selected from the group consisting of hydrogen, sugar, amino sugar, N-alkyl (C1-C6) amino sugar, and acylated amino sugar;
R⁴ is selected from the group consisting of hydrogen, methyl, sugar, amino sugar, N-alkyl (C1-C6) amino sugar, and acylated amino sugar;
R⁵ is selected from the group consisting of hydrogen, methyl, and C2-C6 alkyl;
R⁶ is selected from the group consisting of hydrogen, methyl, sugar, amino sugar, and acylated amino sugar;
R⁷ is selected from the group consisting of hydrogen, methyl, sugar, amino sugar, and acylated amino sugar;
R⁸ is selected from the group consisting of hydrogen, methyl, sugar, amino sugar, and acylated amino sugar;
R⁹ is hydrogen or methyl; and
R¹⁰ is selected from the group consisting of hydrogen, methyl, hydroxyl and amino.

14. A compound according to claim 12 with the following structure: 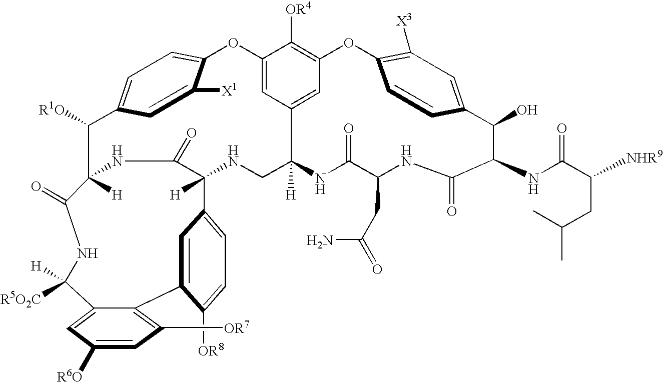
wherein
X¹ is either chloro or hydrogen;
X³ is either chloro or hydrogen;
R¹ is selected from the group consisting of hydrogen and radicals represented by the following structures: 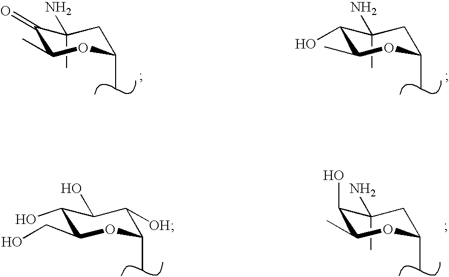
R⁴ is selected from the group consisting of hydrogen, methyl, and radicals represented by the following structures: 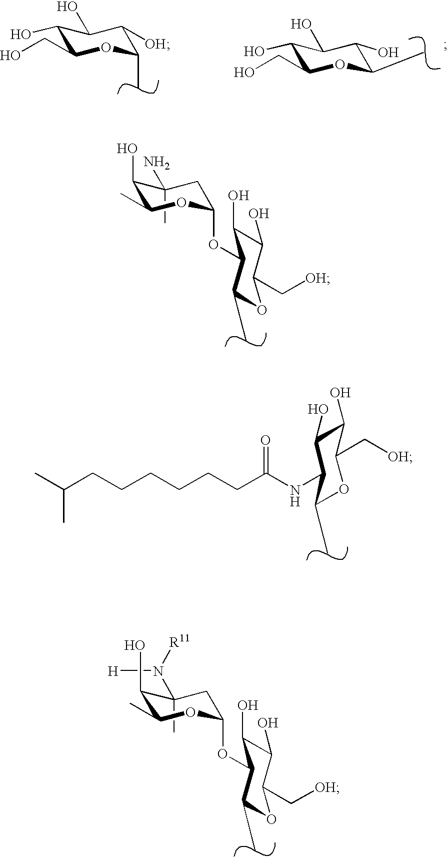
R⁵ is hydrogen or methyl;
R⁶ is hydrogen or methyl;
R⁷ is hydrogen or methyl;
R⁸ is hydrogen or methyl;
R⁹ is hydrogen or methyl;
R¹¹ is selected from the group consisting of radicals represented by the following structures:

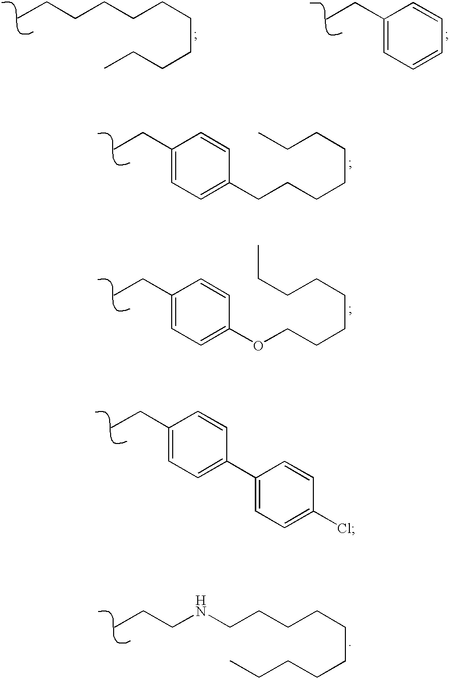

15. A compound according to claim 13 having the following structure: 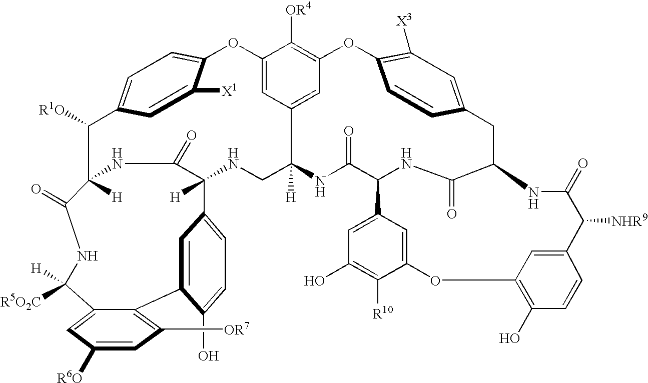
wherein
X¹ is either chloro or hydrogen;
X³ is either chloro or hydrogen;
R¹ is selected from the group consisting of hydrogen, methyl and a radical represented by the following structures: 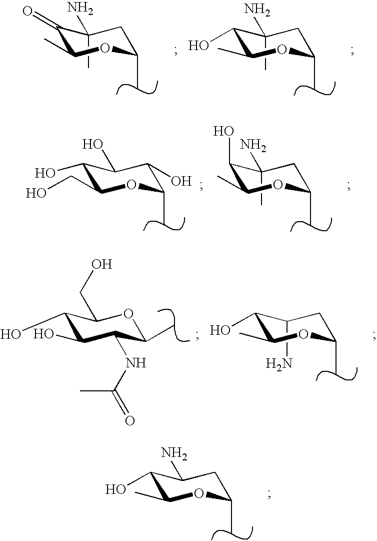
R⁴ is selected from the group consisting of hydrogen, methyl, and a radical represented by the following structures: 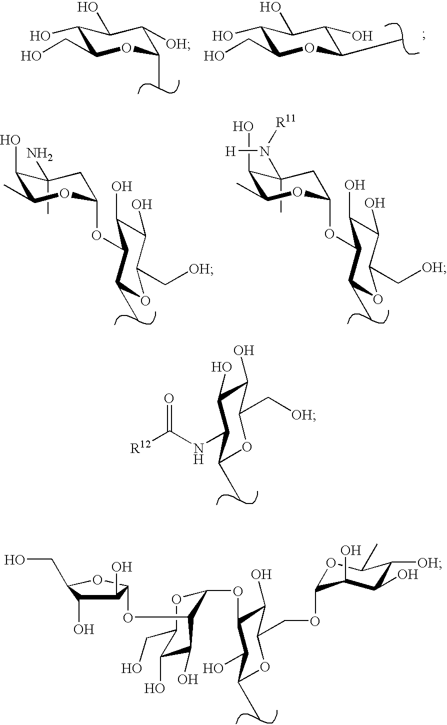
R⁵ is hydrogen or methyl;
R⁶ is hydrogen or methyl;
R⁷ is selected from the group consisting of hydrogen, methyl and a radical represented by the following structures: 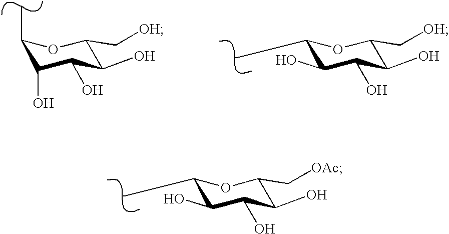
R⁹ is hydrogen or methyl;
R¹⁰ is selected from the group consisting of hydrogen, methyl, hydroxyl, and amino;
R¹¹ is selected from the group consisting of radicals represented by the following structures: 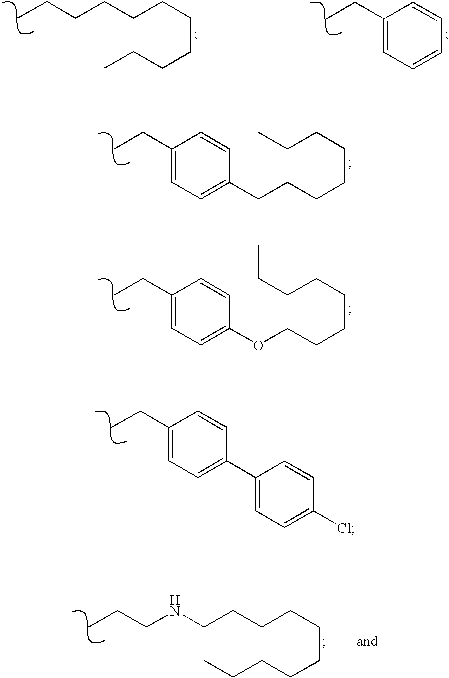
R¹² is selected from the group consisting of hydrogen, methyl, and radicals represented by the following structures:

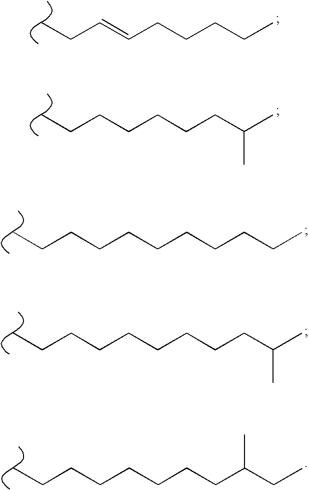

16. A compound of Formula I represented by the following structure: 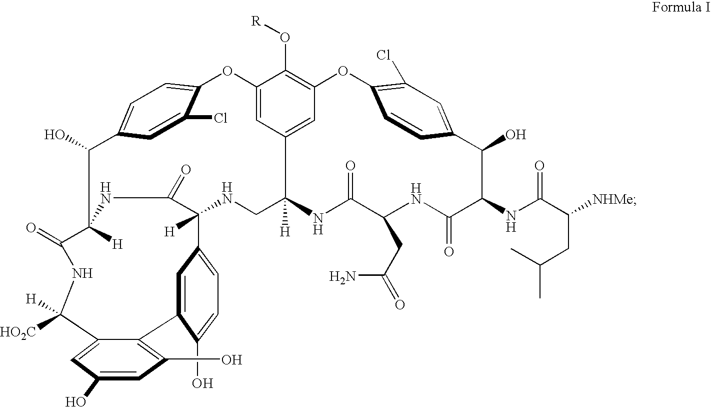
wherein R is selected from the group of radicals consisting of hydrogen, monosaccharide, disaccharide, and trisaccharide; wherein the mono-, di-, and trisaccharides optionally include one or more amino groups and optionally include one or more (C1-C6) alkyls.

17. A compound according to claim 16 wherein R is a disaccharide represented by the following structure:

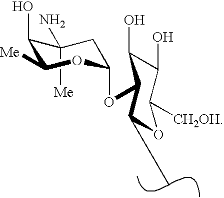

18. A process for converting compound A into compound B where A and B are represented by the following structures: 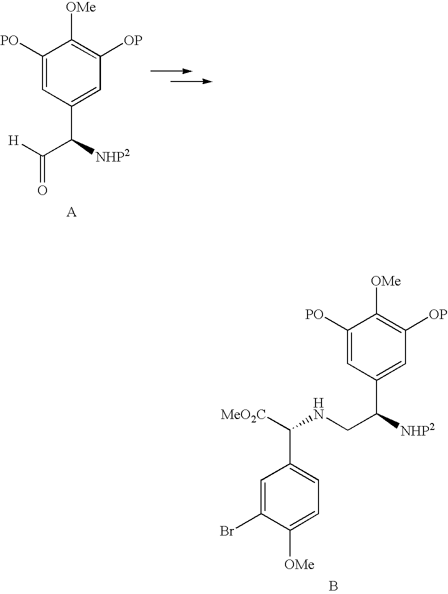
wherein P and P² are protecting groups;
said process comprising the following steps:
Step A: converting compound A to a first intermediate having an imine by reacting the aldehyde of compound A with a second reactant having a primary benzylic amino group for producing the first intermediate; and then
Step B: converting the first intermediate of said Step A to compound B.

19. A process according to claim 18 wherein:
in said Step A: the aldehyde of compound A is reacted with a slight excess of the second reactant and in the presence of a dehydrating agent; and then
in said Step B: the pH of the product of said Step A is adjusted by the addition of glacial acetic acid followed by the addition of a borohydride reagent at a temperature sufficient to allow the reduction of the imine of the first intermediate from step A to be substantially complete after 2 days to give compound B;
wherein:
P is a protecting group for phenols that can be removed in the presence of phenyl methyl ethers, esters, amines protected by P², phenyl bromides and carbamoyl groups; and
P² is a nitrogen protecting group that can be removed in the presence of phenyl chlorides, methyl phenyl ethers, amides, O-MEM groups and benzyl hydroxyl groups.

20. A process for converting compound B into compound C, wherein compounds B and C are represented by the following structures: 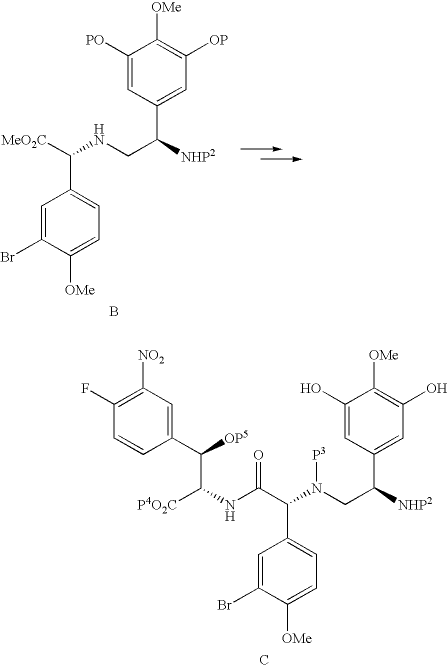
wherein P, P², P³, P⁴, and P⁵ are protecting groups;
said process comprising the following steps:
Step A: converting compound B to a second intermediate having all protected amino groups, unprotected hydroxyls, and an ester group; and then
Step B: converting the second intermediate of said Step A to compound C.

21. A process according to claim 20 wherein:
in said Step A: the free amine of compound B is protected with a protecting group that allows ester hydrolysis, P removal, amide bond formation, Suzuki coupling and diazotization of aniline groups, followed by phenol deprotection by removal of the P protecting groups; and
in said Step B: hydrolyzing the ester group of the second intermediate for revealing a carboxylic acid and forming an amide bond between the carboxylic acid and an ester-protected phenylalanine analog to give compound C;
wherein:
P is a protecting group for phenols that can be removed in the presence of phenyl methyl ethers, esters, amines protected by P², phenyl bromides and carbamoyl groups;
P² is a nitrogen protecting group that can be removed in the presence of phenyl chlorides, methyl phenyl ethers, amides, O-MEM groups and benzyl hydroxyl groups;
P³ is an amine protecting group that is not removed by the reaction conditions listed in steps A and B;
P⁴ is an ester protecting group; and
P⁵ is a hydroxyl protecting group that is not an ester.

22. A process for converting compound C into compound D, wherein compounds C and D are represented by the following structures: 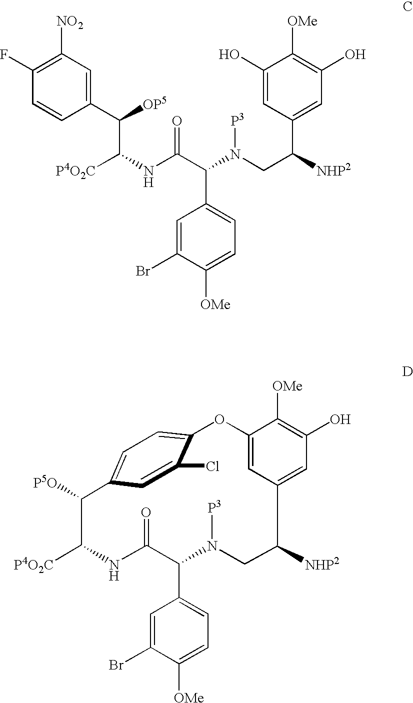
wherein P², P³, P⁴ and P⁵ are protecting groups;
said process comprising the following steps:
Step A: converting compound C to a third intermediate having an aromatic nitro group; and then
Step B: converting the third intermediate of said Step A to compound D.

23. A process according to claim 22 wherein:
in said Step A: compound C is converted to the third intermediate by reaction with a suitable base in the presence of a water scavenging agent at a temperature sufficient for macrocyclization to occur by nucleophilic substitution on the nitro group-bearing ring to give a diphenyl ether functionality followed by separating the two resulting atropdiastereomers; and
in said Step B: the third intermediate is converted to compound D by converting the aromatic nitro group to an amine and then reaction with a diazotizing agent and replacement of the diazo group with a chloro group;
wherein:
P² is a nitrogen protecting group that can be removed in the presence of phenyl chlorides, methyl phenyl ethers, amides, O-MEM groups and benzyl hydroxyl groups;
P³ is an amine protecting group that is not removed by the reaction conditions listed in steps A and B;
P⁴ is an ester protecting group; and
P⁵ is a hydroxyl protecting group that is not an ester.

24. A process for converting compound D and E into compound F, wherein compounds D, E, and F have the following structures: 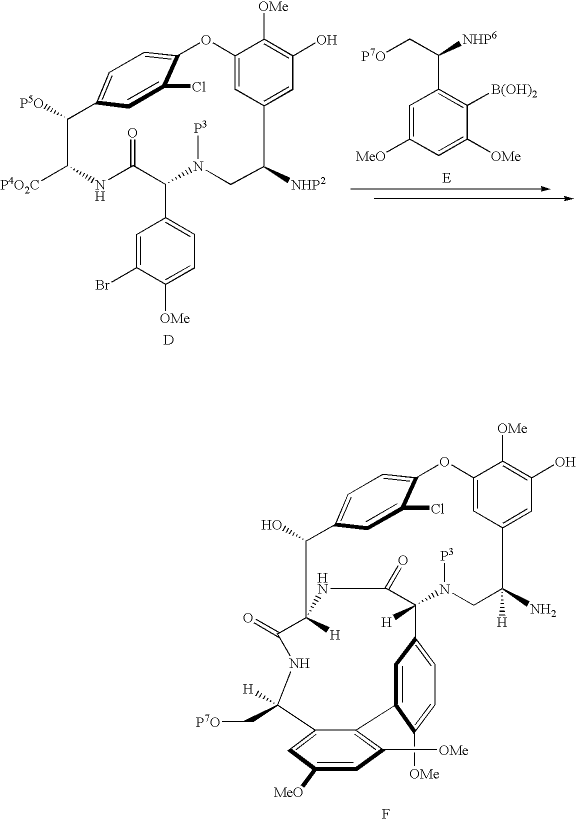
wherein P², P³, P⁴, P⁵, P⁶, and P⁷ are protecting groups;
said process comprising the following steps:
Step A: reacting compounds D and E to form a mixture of atropisomers; then
Step B: isolating one of the desired atropdiastereomers of said Step A; then
Step C: deprotecting the desired atropdiastereomer of said Step B; and then
Step D: converting the deprotected product of said Step C to compound F.

25. A process according to claim 24 wherein:
in said Step A: compounds D and E are mixed in the presence of a suitable catalyst to form a mixture of atropisomers whereby the phenyl ring of compound E is bonded to the phenyl ring of compound D at the carbons that formerly were attached to the boron and bromine, respectively, and separating the atropisomers; and
Step B: isolating the desired atropdiastereomer by heating the undesired atropdiastereomer at a temperature sufficient to convert it to a mixture of atropisomers and again separating the atropisomers; and repeating Step B until a substantial portion of the undesired atropdiastereomer is converted to the desired atropdiastereomer; and
Step C: removing protecting groups P⁵, P⁶ and P⁴ sequentially to give a compound containing a free amino group and a free carboxylic acid; and
Step D: reacting a dilute solution of the compound of step C with a sufficient quantity of amide bond forming reagent to give an intramolecular reaction product; and removal of protecting group P² to afford compound F;
wherein:
P² is a nitrogen protecting group that can be removed in the presence of phenyl chlorides, methyl phenyl ethers, amides, O-MEM groups and benzyl hydroxyl groups;
P³ is an amine protecting group that is not removed by the reaction conditions listed in steps A and B;
P⁴ is an ester protecting group;
P⁵ is a hydroxyl protecting group that is not an ester;
P⁶ is an amino protecting group; and
P⁷ is a hydroxyl protecting group able to be removed in the presence of phenyl methyl ethers and the P³ protecting group.

26. A process for converting compound F into compound G, wherein the compounds F and G are represented by the following structures: 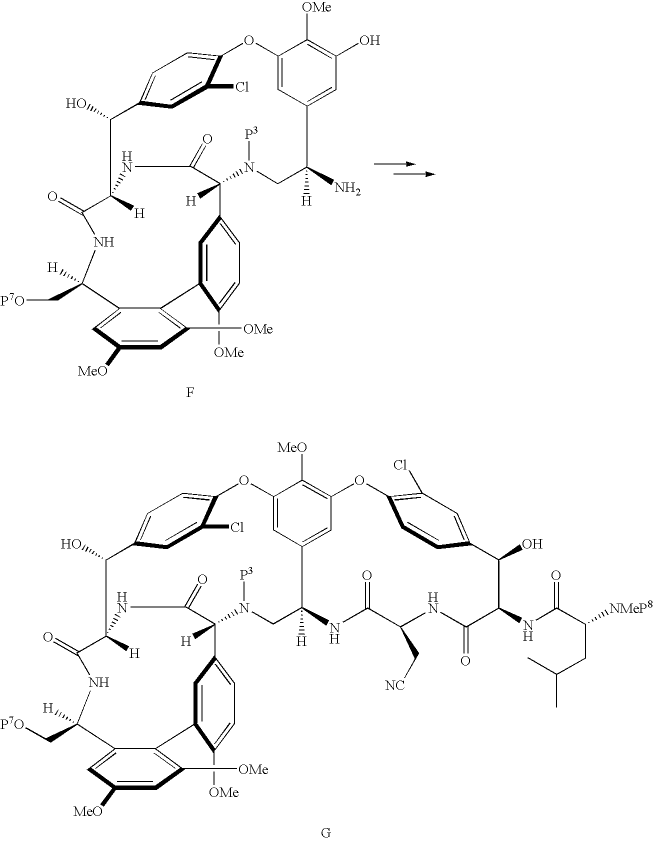
wherein P³, P⁷, and P⁸ are protecting groups;
said process comprising the following steps:
Step A: converting compound F to a fourth intermediate having an amide and possessing the full carbon skeleton of the vancomycin analog; then
Step B: converting the fourth intermediate to a fifth intermediate having a new macrocycle ring possessing a diphenyl ether functionality followed by separation of the desired and undesired atropdiastereomer; and then
Step C: converting the fifth intermediate to compound G.

27. A process according to claim 26 wherein:
Step A: compound F is reacted with a suitably protected tripeptide free carboxylic acid to give the fourth intermediate; then
Step B: the fourth intermediate is treated with a suitable fluoride-containing base in the presence of a water scavenging agent to provide a fifth intermediate; and then
Step C: the aromatic nitro group of the desired atropdiastereomer of the fifth intermediate of said Step B is reduced with a reducing reagent, then the resulting amino group is converted to a diazo group, and then the diazo group is substituted with a chlorine in the presence of a suitable catalyst to give compound G;
wherein:
P³ is an amine protecting group that is not removed by the reaction conditions listed in steps A and B;
P⁷ is a hydroxyl protecting group able to be removed in the presence of phenyl methyl ethers and the P³ protecting group; and
P⁸ is an amino protecting group which is unreactive in said steps A, B and C.

28. A process for converting compound G into compound H, wherein compounds G and H are represented by the following structures: 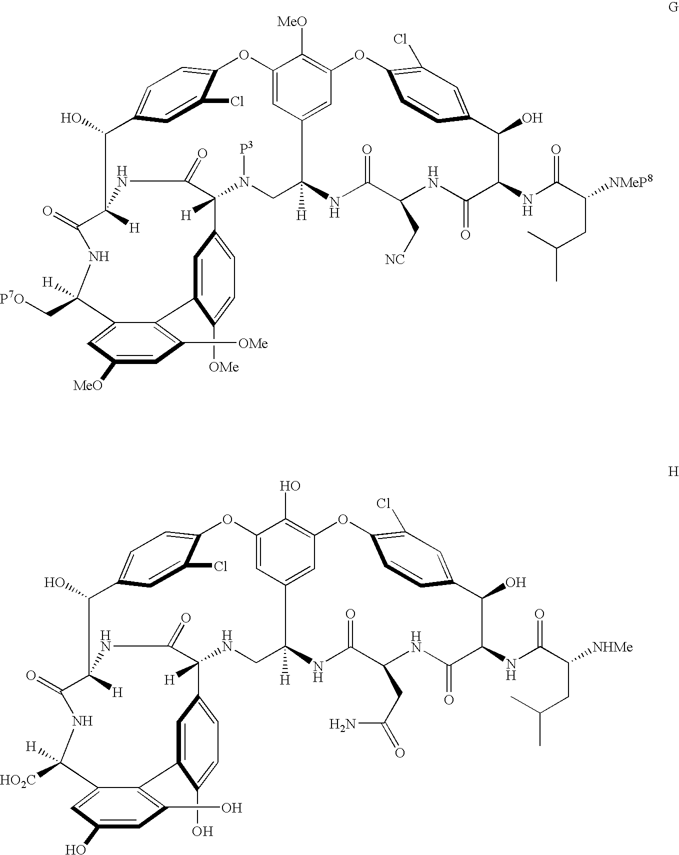
wherein P³, P⁷, P⁸ and P⁹ are protecting groups; said process comprising the following steps:
Step A: converting compound G to a sixth intermediate having a deprotected hydroxyl at P⁷; then
Step B: converting the sixth intermediate of said Step A to a seventh intermediate having carboxylic acid by oxidizing the primary alcohol of the sixth intermediate to form the carboxylic acid; and then
Step C: converting the seventh intermediate of said Step B to compound H by hydrolyzing the cyano group of the seventh intermediate and removing the remaining protecting groups to give compound H.

29. A process according to claim 28 wherein:
in said Step A: the benzylic hydroxyl groups of compound G are protected with protecting group P⁹ and the protecting group P⁷ is removed to form the sixth intermediate; and
in said Step B: the N-methyl group of the sixth intermediate is reprotected with protecting group P⁸ and the primary alcohol from the resulting compound is oxidized to form the carboxylic acid of the seventh intermediate; and
in said Step C: Compound H is formed by hydrolyzing the cyano group of the seventh intermediate of said Step B and the remaining protecting groups P³, methyl ethers, P⁸ and P⁹ are removed to give compound H;
wherein:
P³ is an amine protecting group that is not removed by the reaction conditions listed in steps A and B;
P⁷ is a hydroxyl protecting group able to be removed in the presence of phenyl methyl ethers and the P³ protecting group;
P⁸ is an amino protecting group which is unreactive in said steps A, B and the cyano group hydrolysis of C of claim 7; and
P⁹ is a hydroxyl protecting group that is not removed under the conditions of steps A and B, and the cyano group hydrolysis of step C.
